(19) United States
(12) Patent Application Publication
(10) Pub. No.: US 2017/0340897 A1
(43) Pub. Date: Nov. 30, 2017

(54) APPARATUSES AND METHODS FOR LASER LIGHT THERAPY OF HAIR

(71) Applicant: Martin G. Unger, Toronto (CA)

(72) Inventor: Martin G. Unger, Toronto (CA)

(21) Appl. No.: 15/681,890

(22) Filed: Aug. 21, 2017

Related U.S. Application Data
(63) Continuation-in-part of application No. 14/510,592, filed on Oct. 9, 2014, now Pat. No. 9,737,727.
(60) Provisional application No. 61/937,276, filed on Feb. 7, 2014.

(51) Int. Cl. A61N 5/06; A61N 5/067

(52) U.S. Cl. CPC A61N 5/0617; A61N 2005/067; A61N 2005/0626; A61N 2005/0647

(57) ABSTRACT

Various embodiments are described herein that generally relate to a low-level laser therapy (LLLT) device to aid in at least one of the prevention and treatment of hair loss, rejuvenation of hair, and stimulation of hair regrowth for a certain percentage of users. In at least one embodiment, the device is a laser therapy helmet device, wherein a portion of the device rotates or pivots relative to the treatment surface during use. In at least some embodiments, the device may further comprise a plurality of modes of operation for delivering different amounts of energy to the treatment surface.

### CROSS REFERENCE TO RELATED APPLICATIONS

This application is a continuation in part application of U.S. Non-Provisional patent application Ser. No. 14/510,592 filed on Oct. 9, 2014 which claims the benefit of U.S. Provisional Patent Application No. 61/937,276, filed Feb. 7, 2014. The entire contents of U.S. Non-Provisional patent application Ser. No. 14/510,592 and U.S. Provisional Patent Application No. 61/937,276 are hereby incorporated herein by reference.

### FIELD

The various embodiments described herein relate to apparatuses and methods for laser light therapy of hair, and more particularly relate to apparatuses and methods for a laser light therapy brush and a laser light therapy helmet for the prevention and treatment of hair loss.

### BACKGROUND

Androgenetic alopecia (AGA) or “baldness” occurs in over 80% of the human population during their lifetime. Accordingly, this condition affects hundreds of millions of people worldwide. Over the years, there have been many attempts at treating hair loss with varying results. One of these attempts to treat AGA includes low-level laser therapy (LLLT) with various mixed results.

### SUMMARY

The following is provided to introduce the reader to the more detailed discussion to follow and it is not intended to limit or define any claimed or as yet unclaimed subject matter. One or more groups of claimed or unclaimed subject matter may reside in a combination or a sub-combination of the elements or process steps as described in any part of this document including its claims and figures,

In one broad aspect, in at least one embodiment described herein, there is provided a laser therapy device comprising: an emitter array housing having an active surface with at least one concave shape, the active surface being adapted to face a treatment surface of a user for treating hair loss during use; a plurality of bristles mounted to the emitter array housing and extending outwardly from the active surface; a light therapy module positioned at least partially within the emitter array housing, the light therapy module having: a plurality of emitters for emitting coherent light away from the active surface at a wavelength suitable for treating hair loss; a variable control module coupled to the emitters for controlling the emitters; and a power module coupled to the variable control module for powering the emitters, wherein the bristles and the emitters are arranged in a plurality of rows and in each row the emitters and bristles are arranged substantially linearly.

In at least some of the embodiments, at least one of the rows comprise at least one emitter and at least two bristles arranged on either side of the at least one emitter.

In at least some embodiments, the device comprises a plurality of modes of operation for delivering varying amounts of light energy to the treatment surface of the user, wherein a mode of operation is chosen based on an amount of hair loss experienced by the user to more effectively tailor the treatment to the user's amount of hair loss.

In some embodiments, in a first mode of operation, at least one of the plurality of emitters is controlled to emit coherent light continuously, and at least a second one of the plurality of emitters is controlled to emit coherent light in a pulsed fashion.

In some embodiments, in a second mode of operation the variable control module is configured to sequence the emitters, wherein sequencing the emitters comprises sequentially activating and deactivating rows of emitters, such that at least one band of light energy is emitted along the rows of emitters.

In some embodiments, the sequencing, comprises deactivating at least one row of emitters and activating at least one adjacent row of emitters.

In some embodiments, the at least one band of light energy is emitted along a plurality of adjacent rows of emitters, and wherein sequencing comprises deactivating at least one of the plurality of adjacent rows of emitters and activating at least one row of emitters adjacent to the plurality of rows of activated emitters.

In some embodiments, in a third mode of operation, every emitter in the plurality of emitters is controlled to emit coherent light continuously.

In some embodiments, in a fourth mode of operation, every emitter in the plurality of emitters is deactivated.

In some embodiments, the device further comprises a user interface to allow a user to select between the modes of operation.

In some embodiments, the variable control module further comprises: a control unit for generating control signals; and a switching network coupled between the plurality of emitters and the control unit for receiving the control signals in order to switch at least one emitter between an active state and an inactive state during use according to the mode of operation.

In some embodiments, the housing is shaped as a brush.

In some embodiments, the housing is shaped as a helmet.

In some embodiments, the device comprises a rotational coupling coupled to the emitter array housing; a mount configured to receive a portion of the rotational coupling; and an actuator coupled to the rotational coupling configured to rotate the emitter array housing with respect to the mount during use.

In some embodiments, the actuator is rigidly coupled to the mount or to the emitter array housing.

In some embodiments, the mount comprises a spacer for resting the device on a portion of the user, such that the active surface is in a spaced relationship with the user treatment surface.

In some embodiments, the mount comprises a circumferential guide track and the emitter array housing comprises a guide in mating relationship with the guide track, and wherein in use the actuator displaces the guide to rotate the emitter array housing relative to the mount.

In some embodiments, the housing is spaced vertically from the mount.

In some embodiments, the mount rests on a support, such that the mount supports the weight of the device.

In another broad aspect, in at least one embodiment described herein, there is provided a laser therapy device comprising an emitter array housing having an active surface, the active surface being adapted to face a treatment surface of a user for treating hair loss during use; a light therapy module positioned at least partially within the emitter array housing., the light therapy module having: a plurality of emitters for emitting coherent light away from the active surface at a wavelength suitable for treating hair loss; a variable control module coupled to the emitters for controlling activity of the emitters; and a power module coupled to the variable control module to provide power to the emitters; and a user interface coupled to the light therapy module to allow a user to select from a plurality of modes of operation for delivering varying amounts of light energy to the treatment surface of the user, wherein a mode of operation is chosen based on an amount of hair loss experienced by the user to more effectively tailor treatment to the user's amount of hair loss.

In some embodiments, operating the emitters according to a first sequence comprises deactivating at least one row of emitters and activating at least one adjacent row of emitters.

In some embodiments, operating, the emitters according to a second sequence comprises activating rows of emitters that are not adjacent to one another.

In some embodiments, the plurality of emitters are sequenced to move the at least one light band relative to the active surface during use by activating and deactivating at least one of the plurality of rows of emitters along a common direction wherein the rows of activated emitters are adjacent to one another or are separated by at least one row of deactivated emitters.

In another broad aspect, in at least one embodiment described herein, there is provided a use of a light therapy device defined having a plurality of modes of operation in accordance with the teachings herein, wherein the use comprises: assessing a user's current hair loss; selecting one or more modes of operation for the light therapy device based on the user's current hair loss to deliver an optimum amount of light energy to a treatment surface of the user, such that the use is tailored to the user's amount of hair loss; activating the light therapy device according to one of the one or more modes of operation; and directing the light therapy device towards the treatment surface.

In at least some embodiments, the use further comprises moving the light therapy device across the treatment surface while the device is activated.

In another aspect, a laser therapy device comprising: a helmet housing; an emitter array housing moveably mounted to the helmet housing, the emitter array housing having an active surface being adapted to face a treatment surface of a user for treating hair loss during use; a light therapy module positioned at least partially within the emitter array housing, the light therapy module having: a plurality of emitters for emitting coherent light away from the active surface at a wavelength suitable for treating hair loss; a variable control module coupled to the emitters for controlling the emitters; and a power module coupled to the variable control module for powering, the emitters; at least one actuator coupled to the emitter array housing to move the emitter array housing; and an actuator control module coupled to the at least one actuator and configured to control the at least one actuator to move the emitter array housing with respect to the helmet housing.

In at least some embodiments, the actuator control module is configured to rotate the emitter array housing with respect to the helmet housing.

In at least some embodiments, the actuator control module is configured to pivot the emitter array housing along an arc.

In at least some embodiments, the actuator control module is configured to pivot the emitter array housing about a sagittal plane of a user of the laser therapy device.

In at least some embodiments, the actuator control module is configured to pivot the emitter array housing about a frontal plane of a user of the laser therapy device.

In at least some embodiments, the device comprises first and second emitter array housings and first and second actuators coupled to the first and second emitter array housings respectively, wherein the first actuator is configured to moving the first emitter array housing in a front-back pivoting manner and the second actuator is configured to move the second emitter array housing in a side-to-side pivoting manner.

In at least some embodiments, the first emitter array housing is located at a rear portion of the helmet housing and the second emitter array housing is located at a front central portion of the helmet housing.

In at least some embodiments, the second emitter array housing is located at a rear portion of the helmet housing and the first emitter array housing is located at a front central portion of the helmet housing.

In at least some embodiments, the device comprises first, second and third emitter array housings and first, second and third actuators coupled to the first, second and third emitter array housings respectively, wherein the first actuator is configured to moving the first emitter array housing in a front-back pivoting manner at a rear portion of the helmet, the second actuator is configured to move the second emitter array housing in an arcing motion along the right side of the helmet housing and the third actuator'is configured to move the third emitter array housing in an arcing motion along the left side of the helmet housing,

In at least some embodiments, the actuator comprises a motor and the device comprises a coupling for coupling an output shaft of the motor to the light emitter array housing.

In at least some embodiments, the actuator comprises a motor and the device comprises a guide wire that is disposed along a portion of the helmet housing defining a pivot arc along and a guide wheel that is coupled to the motor and to the light emitter array housing, and the guide wheel engages the guide wire wherein during use the motor rotates the guide wheel which moves along the guide wire causing the light emitter array to pivot along the arc.

In at least some embodiments, the actuator comprises a motor and the device comprises a guide track that is disposed along a portion of the helmet housing defining a pivot arc along and a guide wheel that is coupled to the motor and to the light emitter array housing, and the guide wheel engages the guide track wherein during use the motor rotates the guide wheel which moves along the guide track causing the light emitter array to pivot along the arc.

In at least some embodiments, the actuator control module is configured to vary at least one of the speed and direction of movement of the emitter array housing.

In at least some embodiments, the device comprises a control unit that is configured to control the emitter array housing to operate according to one of a plurality of modes of operation for delivering varying amounts of light energy to the treatment surface of the user, wherein a mode of operation is chosen based on an amount of hair loss experienced by the user to more effectively tailor the treatment to the user's amount of hair loss.

In at least some embodiments, the control unit is configured to deliver varying amounts of light energy according to the position of the emitter array housing relative to a portion of the treatment surface of the user.

In at least some embodiments, the control unit is configured to deliver a larger amount of light energy when the position of the emitter array housing faces a portion of the treatment surface of the user with more hair than other portions of the treatment surface of the user.

In at least some embodiments, the control unit is configured to deliver a smaller amount of light energy when the position of the emitter array housing faces a portion of the treatment surface of the user with less hair than other portions of the treatment surface of the user.

In at least some embodiments, in a mode of operation the variable control module is configured to sequence the emitters, wherein sequencing the emitters comprises sequentially activating and deactivating rows of emitters, such that at least one band of light energy is emitted along the rows of emitters.

In at least some embodiments, the sequencing comprises deactivating at least one row of emitters and activating at least one adjacent row of emitters.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### DRAWINGS

For a better understanding of the various embodiments described herein, and to show more clearly how these various embodiments may be carried into effect, reference will be made, by way of example, to the accompanying drawings which show at least one example embodiment and the figures will now be briefly described.

FIG. 1 is a bottom view of an example embodiment of a laser therapy brush device;

FIG. 2 is a cross-sectional side view of the laser therapy brush device of FIG. 1;

FIG. 3 is another cross-sectional view of the laser therapy brush device of

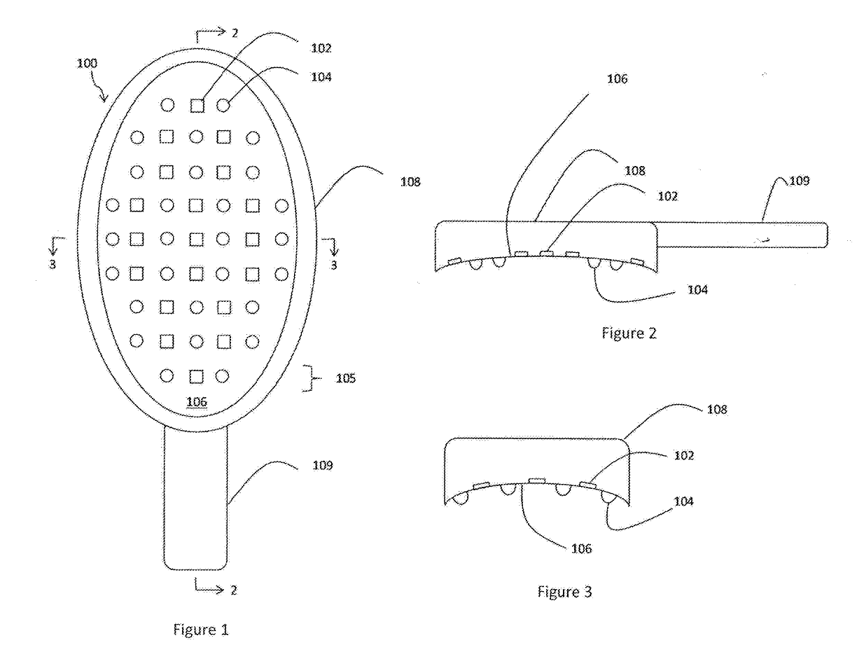

FIG. 1;

FIG. 4 is a cutaway bottom view of the laser therapy brush module of FIG. 1;

FIG. 5 is a cutaway bottom view of an alternative embodiment of the laser therapy brush module of FIG. 1;

FIGS. 6A-6C illustrate an example of a sequential activation mode of operation in which a band representing activated laser emitters propagates through the rows of emitters over time for the laser therapy brush module of FIG. 1;

FIG. 7 is a perspective view of an example embodiment of a laser therapy helmet;

FIG. 8 is a plan view of an example embodiment of a laser therapy helmet emitter array that may be used with the laser therapy helmet of FIG. 7;

FIG. 9 is a cross-sectional side view of the laser therapy helmet of FIG. 7;

FIG. 10 is a cross-sectional front view of the laser therapy helmet of FIG. 7;

FIG. 11 is a bottom view of the laser therapy helmet of FIG. 7;

FIG. 12 is a perspective view of an example embodiment of a laser therapy helmet with a guide track;

FIG. 13 is a perspective view of an example embodiment of a laser therapy helmet with a guide wire;

FIG. 14 is a perspective view of an example embodiment of a ground-coupled or suspended laser therapy helmet;

FIG. 15A is a top view of an example of an alternative embodiment of a laser therapy helmet emitter array housing;

FIG. 158 is a top view of an example of another alternative embodiment of a laser therapy helmet emitter array housing;

FIGS. 16A-16D are perspective views of examples of other alternative embodiments of a laser therapy helmet with an automated pivoting emitter array housing;

FIGS. 17-18 are perspective views of examples of other alternative embodiments of a laser therapy helmet with two or more automated pivoting emitter array housings.

FIG. 19 is a cutaway bottom view of a laser therapy helmet module;

FIG. 20, in a sectional front view, illustrates an example embodiment of a variable laser therapy helmet module.

FIGS. 21A-21C illustrate an example of a sequential activation mode of operation in which a band representing activated laser emitters propagates through the rows of emitters over time for a laser therapy helmet;

FIG. 22A is a bottom view showing an example mode of operation for a laser therapy brush device that comprises at least one pulsing laser emitter and at least one continuous laser emitter;

FIG. 228 is a plan view showing an example mode of operation for a laser therapy helmet that comprises a plurality of pulsing laser emitters and a plurality of continuous laser emitters;

FIG. 23 is a block diagram of an example embodiment of a laser emitter module that may be used with a laser therapy brush or helmet device described herein;

FIG. 24 is a block diagram of the laser emitter module of FIG. 23 when the laser emitters are configured as groups of pulsed emitters and continuous emitters; and

FIG. 25 is a block diagram of the laser emitter module of FIG. 23 when the laser emitters are grouped in rows for sequential activation.

Further aspects and features of the embodiments described herein will appear from the following description taken together with the accompanying drawings.

### DESCRIPTION OF VARIOUS EMBODIMENTS

Various apparatuses or processes will be described below to provide an example of at least one embodiment of claimed subject matter. No embodiment described below limits any claimed subject matter and any claimed subject matter may cover processes, apparatuses, devices, or systems that differ from those described below. The claimed subject matter is not limited to apparatuses, devices, systems, or processes having all of the features of any one apparatus, device, system, or process described below or to features common to multiple or all of the apparatuses, devices, systems, or processes described below. It is possible that an apparatus, device, system, or process described below is not an embodiment of any claimed subject matter. Any subject matter that is disclosed in an apparatus, device, system, or process described below that is not claimed in this document may be the subject matter of another protective instrument, for example, a continuing patent application, and the applicants, inventors, or owners do not intend to abandon, disclaim, or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term such as, but not limited to, 1%, 2%, 5%, or 10%, for example, if this deviation would not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount such as, but not limited to, 1%, 2%, 5%, or 10%, for example, of the number to which reference is being made if the end result is not significantly changed.

As used herein, the wording “and/or” is intended to represent an inclusive-or. That is, “X and/or Y” is intended to mean X or Y or both, for example. As a further example, “X, Y, arid/or Z” is intended to mean X or Y or Z or any combination thereof.

Androgenetic alopecia (AGA) or “baldness”, often referred to as Male Pattern Hair Loss (MPHL) in men and Female Pattern Hair Loss (FPHL) in women, occurs in over 80% of the human population during their lifetime (Unger, 2003). In European men, for example, AGA affects between 29-40% of men aged 18-59 years old, with 30% of men aged 35 or older reporting “moderate hair loss” (Budd, 2000). In the U.S., hair loss occurs in 53% of men by age 40-49, and in 40% of women by age 40 (Leavitt, 2006). In this description, the terms AGA, hair loss, baldness, MPHL and FPHL may be used interchangeably.

Low Level Light Therapy (LLLT) is an emerging technology that has been developed to prevent hair loss, stimulate hair regrowth in areas of hair loss and strengthen hair for a certain percentage of users (Unger, 2003). The inventor, who has long been involved with the development of LLLT both as a Medical Director and a Medical Consultant for several companies that develop light therapy products, has found that while an optimal dosage of LLLT will usually lead to bio-stimulation having the above-described beneficial effects on a user's hair, overstimulation will not lead to any beneficial effects and may actually have detrimental effects. Overstimulation can be caused by treatment with devices having, too many laser diodes, or alternately by treatments that are too long or too frequent. The inventor has found that it is beneficial to get the right amount of energy to the user's treatment surface and that it is beneficial to part the user's hair such that the LLLT energy is properly delivered to the user's treatment surface, which will usually be the scalp, but may be different areas for different users. The various embodiments described herein relate to LLLT devices and methods for providing an optimal amount of LLLT to a user's treatment surface, such that bio-stimulation results without causing overstimulation.

Reference is now made to FIGS. 1 to 3, which illustrate an embodiment of a laser therapy brush device 100. The brush device 100 comprises an emitter array housing 108, an emitter array active surface 106, a plurality of laser emitters 102, a plurality of bristles 104 and a handle 109. The emitter array active surface 106 is a concave surface of the housing 108. The emitters 102 and the bristles 104 are mounted to the housing 108 such that the bristles 104 extend outwardly from the active surface 106, and such that the emitters 102, when activated, emit coherent light away from the active surface 106. In at least some embodiments, each emitter 102 emits coherent light outwardly and approximately perpendicularly away from the active surface 106.

The bristles 104 and the emitters 102 are arranged in a plurality of rows wherein at least one of the rows comprises at least one emitter 102 and at least two bristles 104 arranged on either side of the at least one emitter 102 in a substantially linear fashion. For example, row 105 illustrates a particular embodiment of a row. In some embodiments, in each row, each emitter 102 is aligned with two adjacent bristles 104, such that the bristles 104 and the emitters 102 alternate in each row, as illustrated in the example embodiment shown in FIGS. 1 to 3. Henceforth this positioning is referred to as an “aligned alternating arrangement”. Although the illustrated embodiments of the brush device 100 illustrate the rows as being perpendicular to the handle 109, a person of ordinary skill in the art would understand that the rows could also be arranged to be parallel to the handle 109, or angled diagonally to the handle 109 in alternative embodiments. Furthermore, although the active surface 106 and housing 108 are shown as being shaped ovally, a person of ordinary skill in the art would understand that the active surface 106 and housing 108 may be otherwise shaped, such as having a circular, hexagonal or rectangular shape in alternative embodiments.

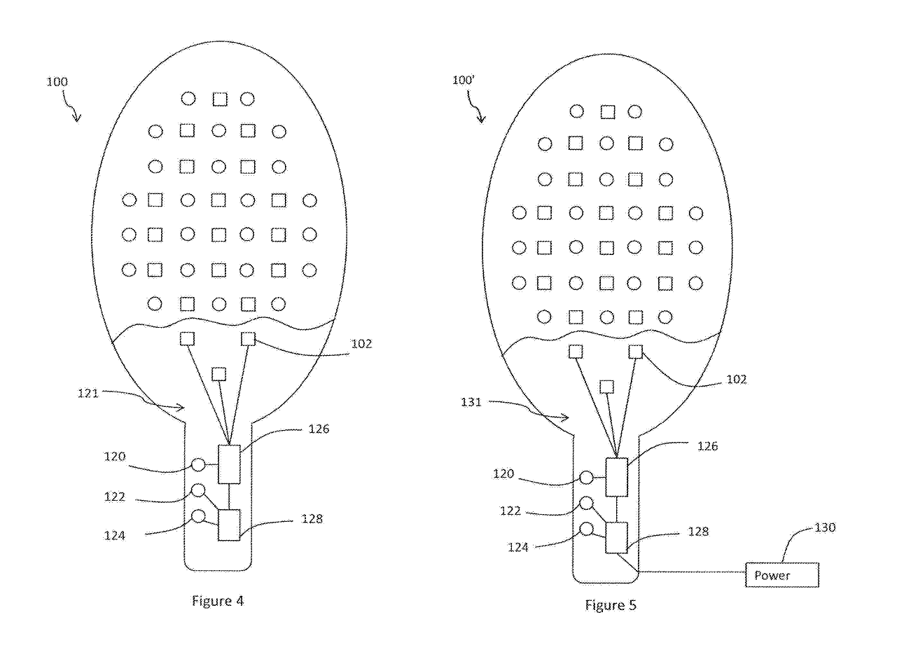

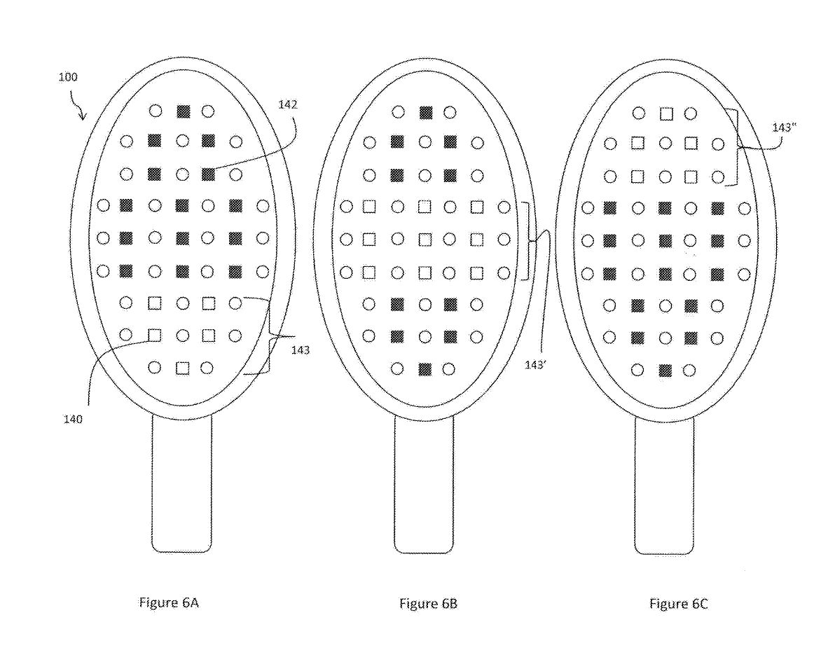

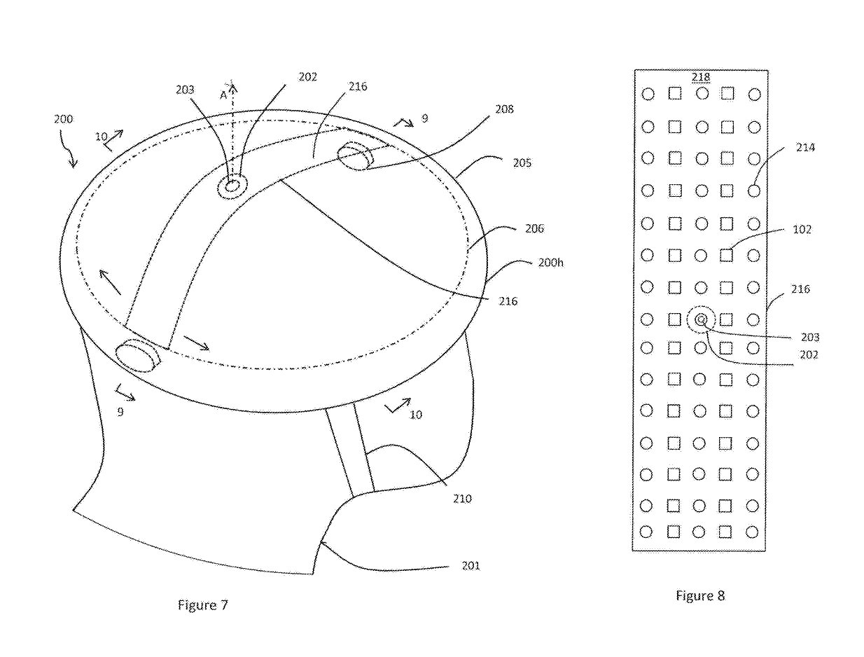

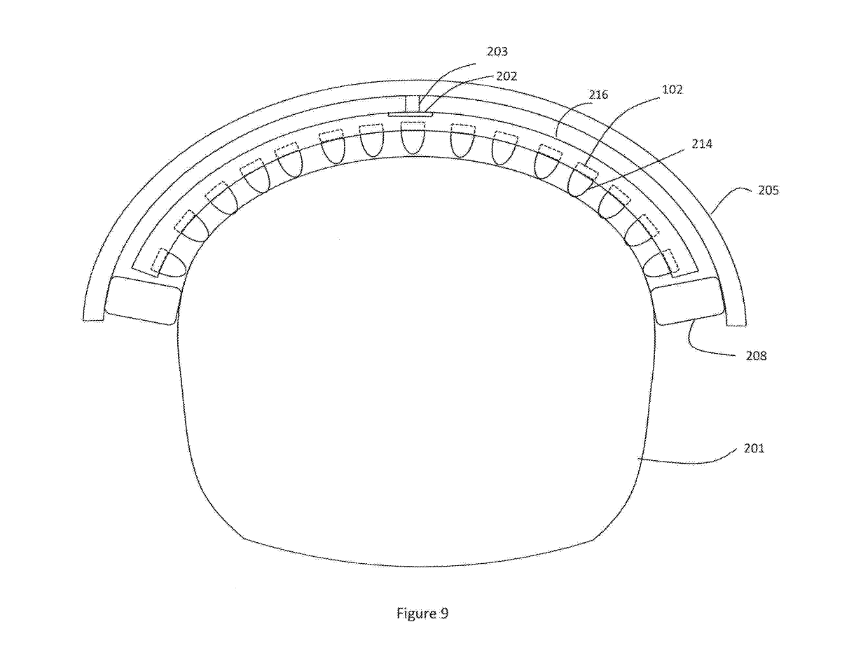

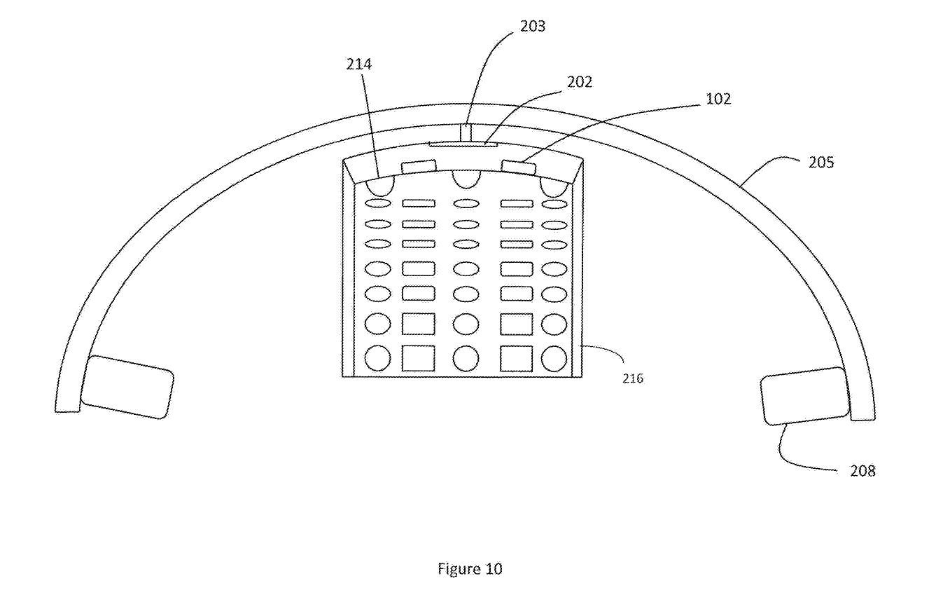

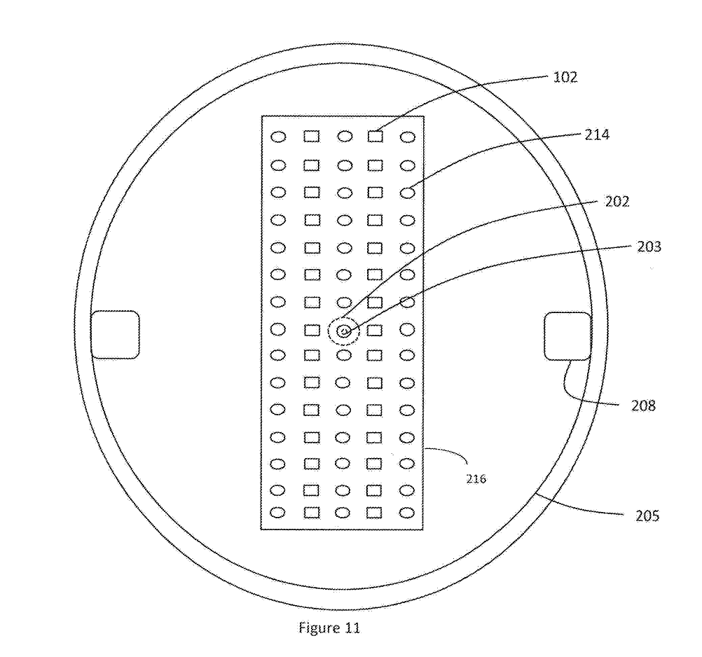

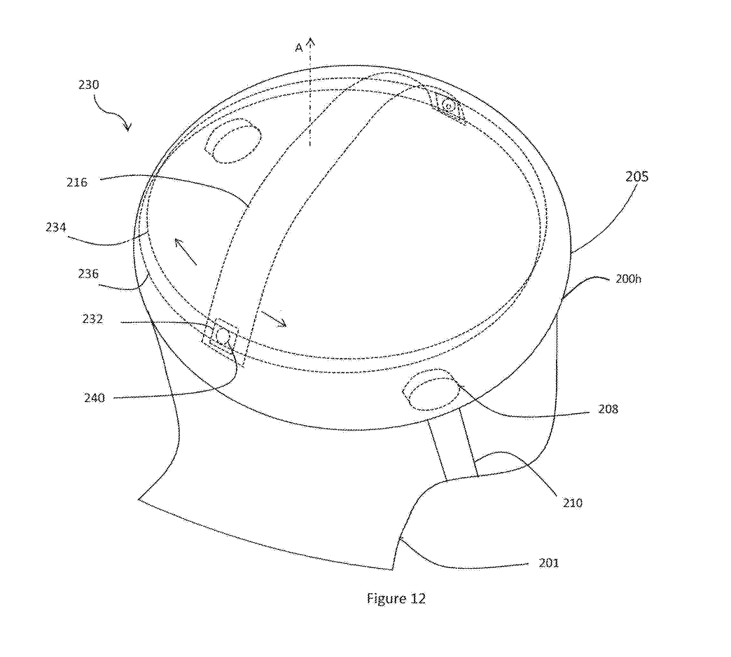

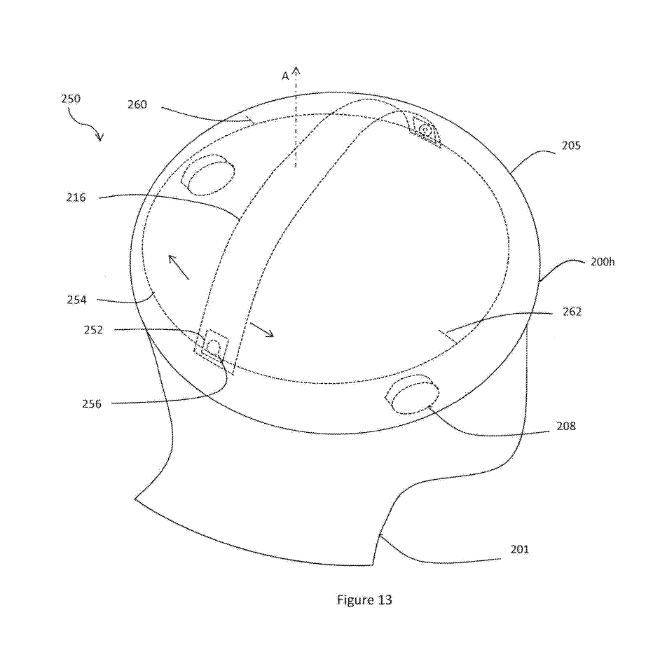

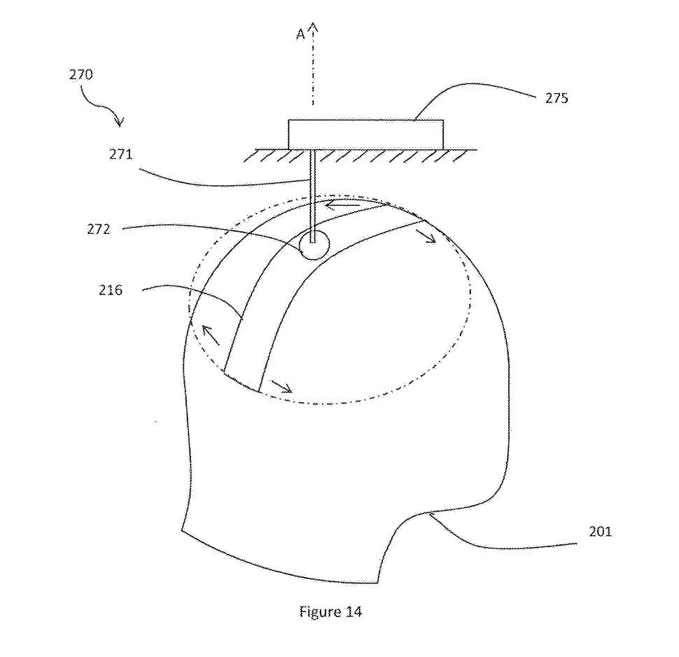

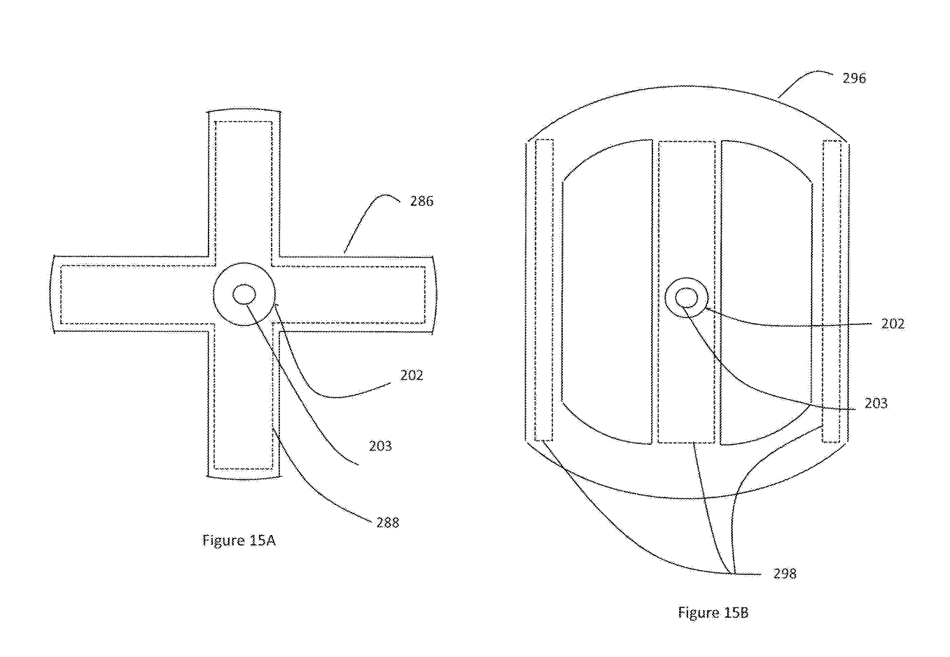

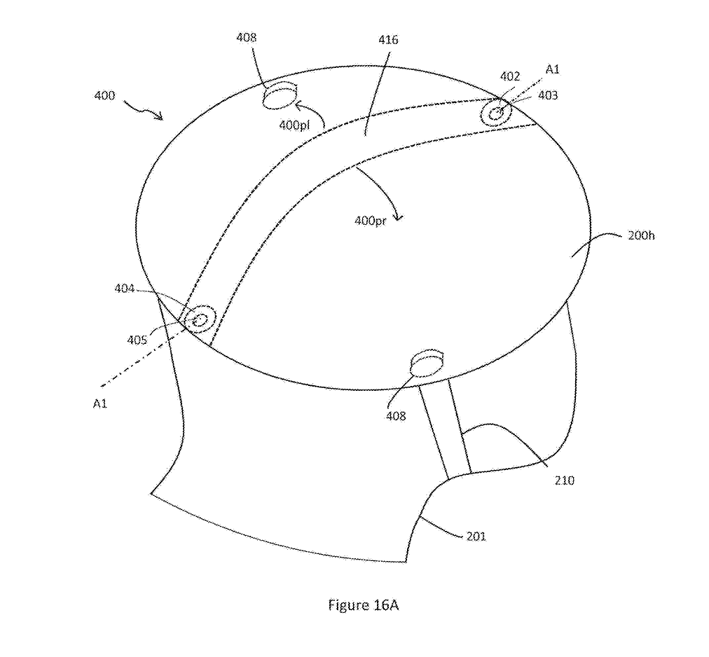

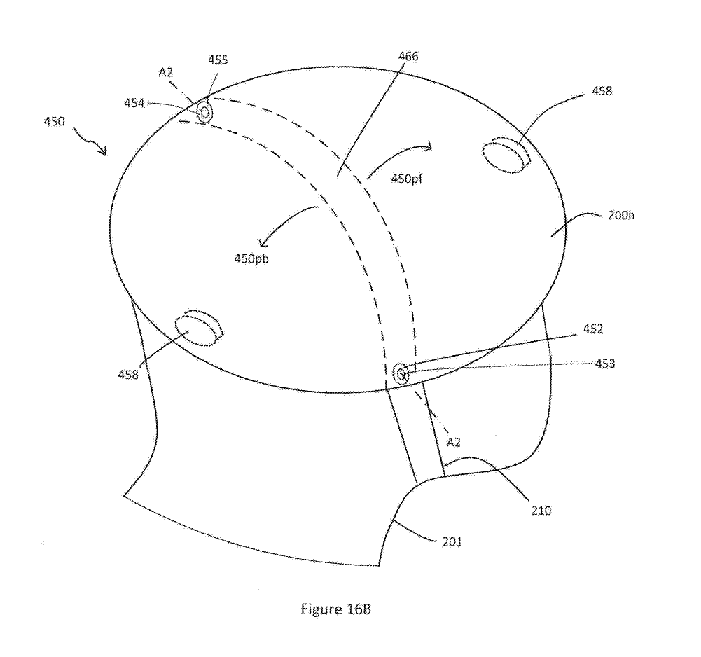

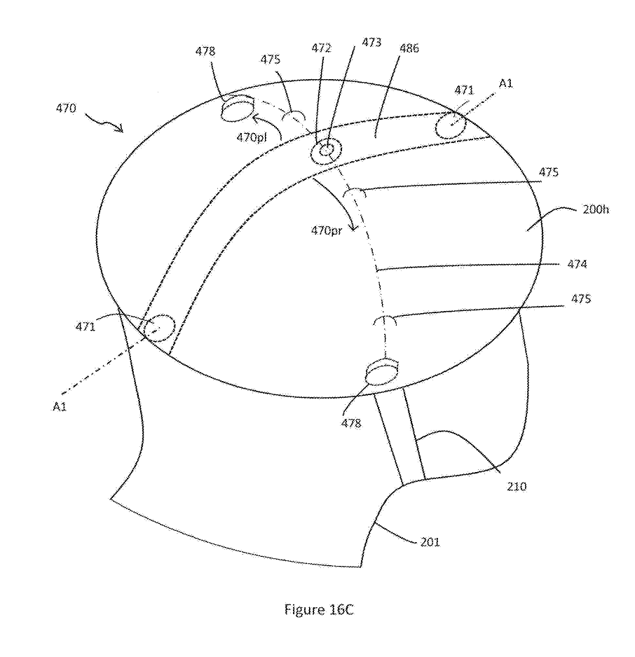

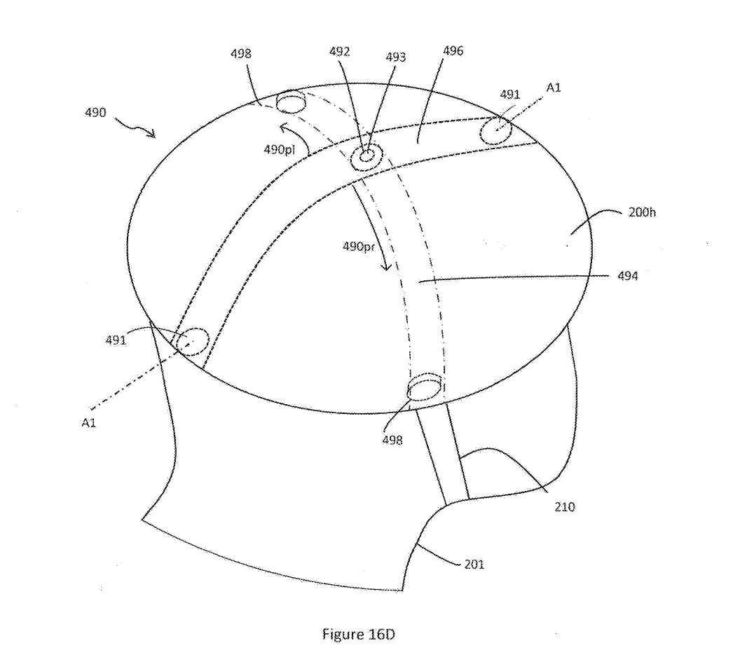

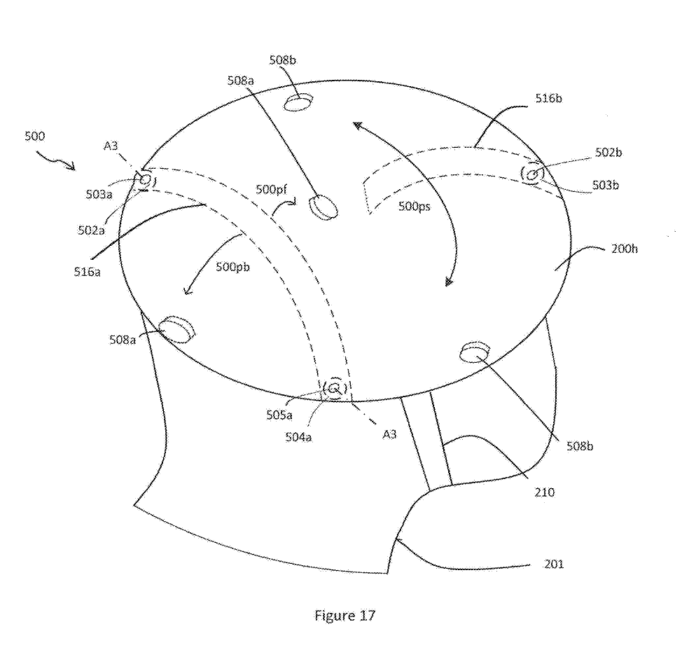

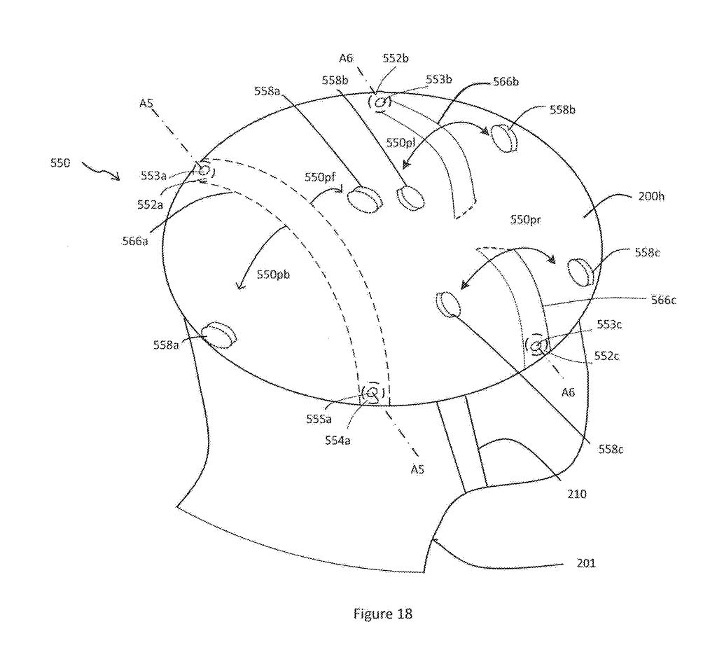

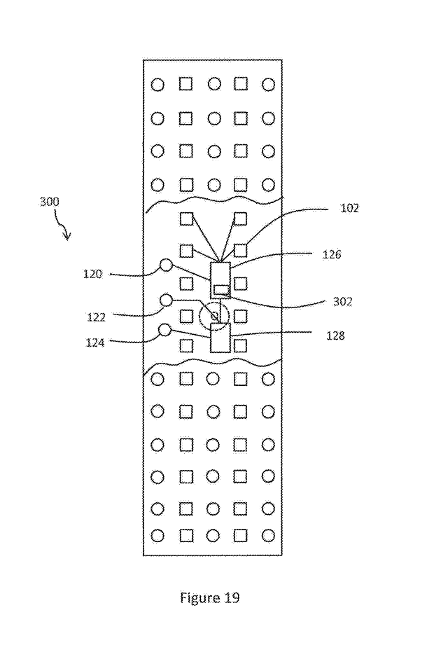

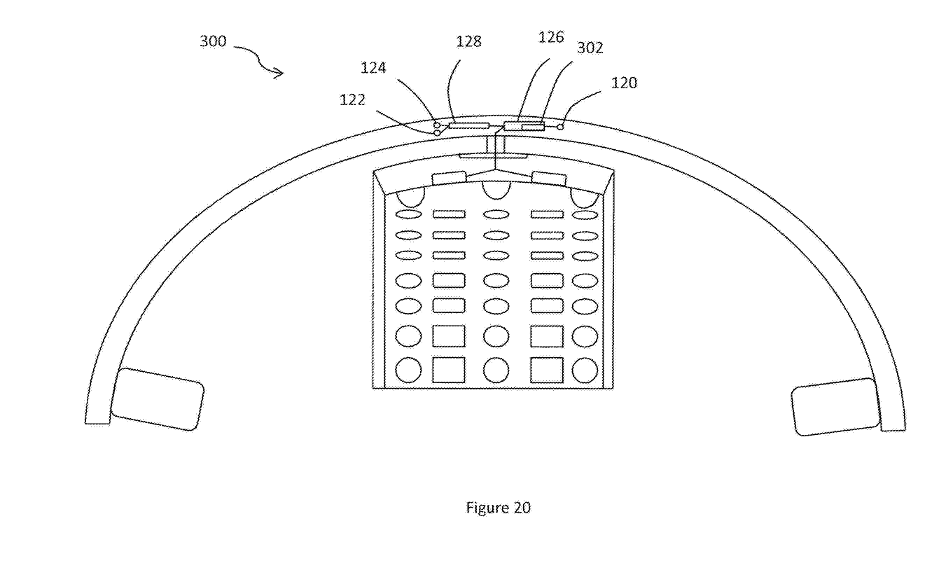

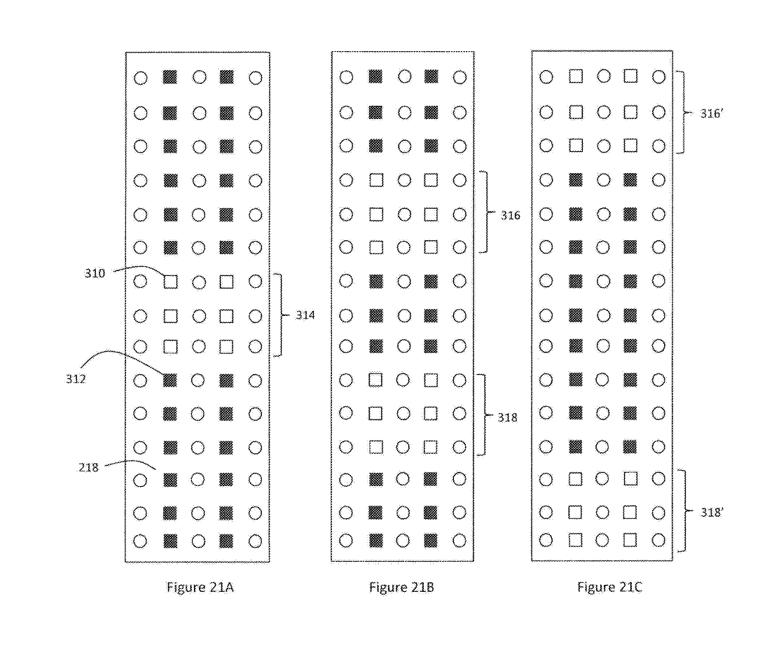

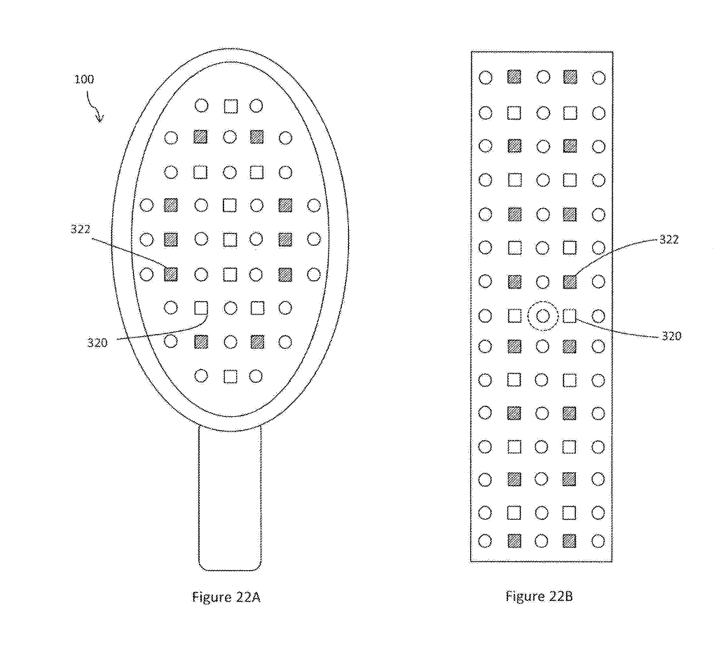

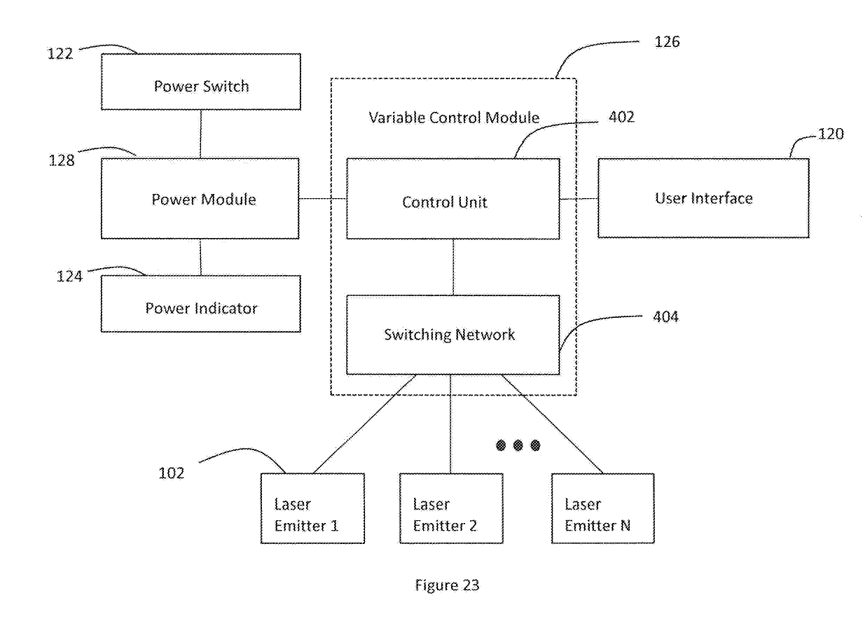

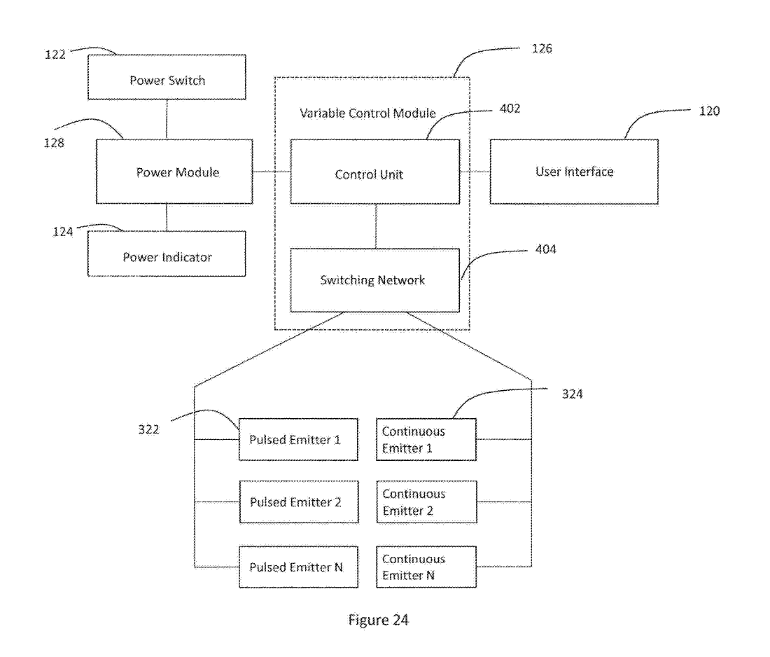

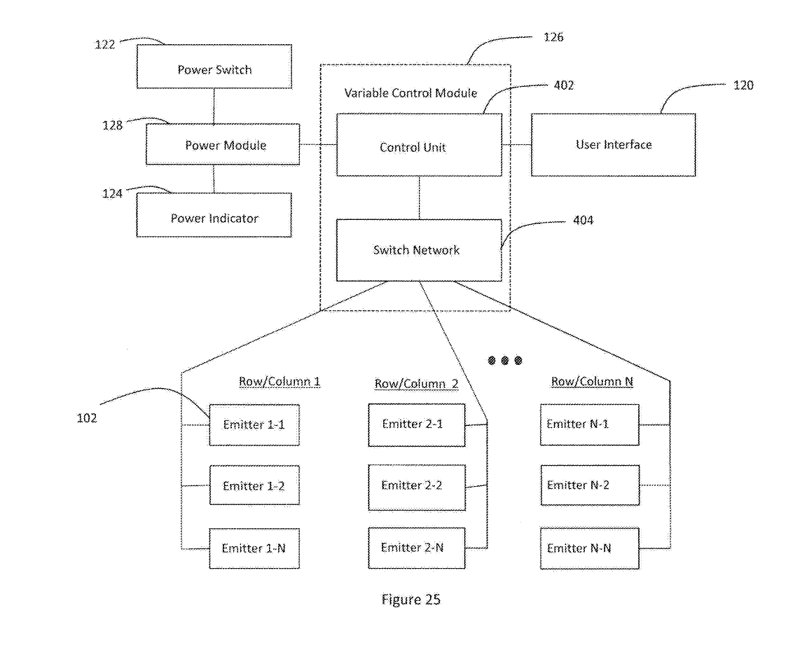

Referring specifically to FIGS. 2 and 3, shown therein are sectional side and end views of the brush device 100. These views show more clearly that the active surface 106 may be concave along two axes, the major and minor axes, such that it is shaped for positioning against a user's treatment surface that may have a convex shape, such as a user's scalp, for example.

In use, the user activates the brush device 100 and positions the active surface 106 against the user's desired treatment surface. The user then moves the brush device 100 across the treatment surface in a direction collinear with the rows of emitters 102 and bristles 104. Alternatively, the user can simply hold the brush device 100 above the treatment surface. In embodiments where the bristles 102 and emitters 104 are arranged in the aligned alternating arrangement, as the brush device 100 moves across the treatment surface, each bristle 104 is closely followed by an emitter 102, which is closely followed by another bristle 104. When used on the user's scalp, this aligned alternating arrangement ensures that the bristles 104 part the user's hair in advance of each emitter 102, such that energy is directly delivered to the treatment surface, instead of being absorbed by the user's hair. Further, the concave shape of the active surface 106 ensures a comfortable mating of the active surface of the brush device 100 with the user's treatment surface, as well as ensuring that the emitters 102 emit coherent light approximately perpendicularly to the user's treatment surface when active in order to optimally deliver energy to the user's treatment surface. Other angles may be used in alternative embodiments where applicable,

The brush device 100 is typically used for the treatment of AGA such that the user's treatment surface is the area of a user's head that experiences hair growth, such as the scalp. In this description, the terms scalp, head or treatment surface may be used interchangeably. However, a person of ordinary skill in the art will understand that other convex surfaces of a user's body may be treated by LLLT; for example, the brush device 100 may be positioned against a user's eyebrow, chin, jawbones, chest, arms or legs.

In most embodiments, the housing 108 may be fabricated from material known to those of ordinary skill in the art as having sufficient rigidity to avoid unwanted flexing during use. In some embodiments, the housing 108 may be made of plastic, rubber, or metal,

In most embodiments, the bristles 104 are sized and shaped to part the user's hair in advance of emitters 102 without creating discomfort for the user. In most embodiments, the bristles 104 will be blunt and will be sized to provide sufficient rigidity to optimally part, the user's existing hair in advance of the emitters 102 passing over the treatment surface when the brush device 100 is in use (as described above). In an example embodiment, the bristles 104 may have a height of about ½ an inch, although other dimensions may be used. The bristles 104 may be constructed of a material providing sufficient rigidity to part a user's hair. In some embodiments, the bristles may be made of rubber, plastic, or metal.

In most embodiments, the laser emitters 102 are laser diodes. A person of ordinary skill in the art would understand that other emitters of coherent light may be used. Throughout this description, the terms laser light or coherent light are used interchangeably. In many embodiments, the laser emitters may be configured to output low-level laser light at a wavelength in the range of 600 nm to 1000 nm. However, a person of ordinary skill in the art will understand that the laser emitters may be configured to emit light at other wavelengths suitable for treating hair loss. In particular, the emitters are chosen such that during use enough energy is supplied to the user treatment surface for bio-stimulation, but not so much as to cause overstimulation. In some embodiments, each laser emitter 102 may be configured to output light energy having 1 to 5 mW of power, for example.

In at least some embodiments, the laser emitters 102 may be configured to output 5 mW of power at a wavelength of 650 nm,

In at least some embodiments, 10-30 laser emitters may be mounted to the housing 108 of the brush device 100. For example, in the illustrated embodiment of FIG. 1, 19 emitters 102 are mounted to the housing 108,

Reference is now made to FIG. 4, which illustrates an example embodiment of a laser therapy module 121 that may be used with the laser therapy brush device 100. The laser therapy module 121 comprises the emitters 102; a variable control module 126 coupled to the emitters 102 for controlling the emitters 102; and a power module 128 coupled to the variable control module 126 for powering the emitters 102. The brush device 100 may further comprise at least one of a user interface 120, a power switch 122, and a power indicator 124. Some or all of these elements may be mounted on one or more printed circuit boards, which may or may not be flexible, and are located in the handle portion 109 and possibly part of the housing 108 of the brush device 100.

The variable control module 126 is generally operable to selectively activate, deactivate or otherwise control individual emitters 102 or emitter groups (e.g. rows of emitters, columns of emitters, or user-determined or predetermined groups of emitters that are grouped together and can be controlled together). In some embodiments, the variable control module 126 comprises a switching network (described in relation to FIGS. 23-25) for activating or deactivating the emitters 102. In such embodiments, the variable control module 126 activates or deactivates the emitters 102 by sending control signals to switches within the switching network. Individual emitters 102 or emitter groups can be coupled to switches in the switching network for individual or group control by the variable control module 126 via control signals. The variable control module 126 also comprises a processor, which can be programmed or otherwise configured to provide control signals. The variable control module 126 is generally provided with software instructions, that when executed by the processor configures the processor to implement certain functionality. It should be noted that in alternative embodiments other electric components may be used to implement the functionality of the processor such as, but not limited to, an Application. Specific Integrated Circuit (ASIC), a Field Programmable Gate Array (FPGA), discrete electronics and the like; these alternative elements and the processor are collectively referred to as a controller.

In at least some embodiments, the variable control module 126 may be operable to provide control signals in order to control the total length of treatment time and therefore the total emission power of the emitters 102, individually or in emitter groups. Treatments lasting 12-15 minutes given three times a week or on alternate days are desirable and preferred.

In at least some embodiments, the variable control module 126 is operable to activate, deactivate or otherwise control the emitters 102 or emitter groups for user-determined or predetermined periods of time. In such embodiments, the variable control module 126 may be operable to activate and/or deactivate or otherwise control the emitters 102 or emitter groups according to user-determined or pre-determined timed sequences and/or durations of time (to limit treatment time).

Accordingly, it should be understood that in at least some embodiments the variable control module 126 has control over the activation, deactivation, and timing for laser light emitted by the emitters.

However, in at least some embodiments the variable control module 126 may only have limited control over the emitters 102. For example, in some embodiments, the variable control module 126 may be operable to only control the activation and deactivation of the emitters 102 or the emitter groups.

The user interface 120 may be coupled to the variable control module 126 to allow the user to select a mode of operation of the brush device 100. In some embodiments, each mode of operation delivers a different amount of light energy to the user's treatment surface. Accordingly, a user may select a mode of operation based on an amount of hair loss experienced by the user in order to more effectively treat the hair loss for that particular user. This is beneficial since hair blocks at least some light energy from reaching the user's treatment surface, and therefore if a user has less hair, it may be beneficial to select a mode of operation wherein less energy is output to the user's treatment surface in order to prevent overstimulation.

In at least some embodiments, the user interface 120 is operable to select between four modes of operation including a first mode in which all emitters are deactivated; a second mode in which all emitters are activated and emit light continuously; third mode in which at least one emitter of the plurality of emitters emits coherent light in a pulsed fashion; and a fourth mode in which the variable control module 126 sequentially activates and deactivates rows of emitters, such that at least one band of light energy propagates along the rows of emitters.

As in most embodiments the mode of sequential activation emits less total energy than the mode with a combination of pulsing and continuous emitters. The mode of sequential activation is best suited for patients with a small amount of remaining hair (thin hair) to prevent overstimulation, whereas the mode with a combination of pulsing and continuous emitters is best suited for patients with moderate hair loss. As the mode with the continuous emitters produces more total output energy (e.g. 5 mW of output power for each of 19 diode lasers provides 95 mW of output power), than the mode with a combination of pulsing and continuous emitters (e.g. 50 mW of output power from 10 continuous 5 mW emitters, and 22.5 mW of output power from 9 pulsed 5 mW emitters operating only half the time, provides 72.5 mW of total output power), the mode with continuous emitters is best suited for patients with early or minimal hair loss.

In this way the various modes of treatment allow for tailoring of the total energy and power output to each individual during a treatment session, which results in better-controlled bio-stimulation without overstimulation. These modes of operation are further discussed with respect to FIGS. 6, and 21A-25. It should be understood that these modes of operation may apply to any of the various embodiments of the brush device 100 and/or a helmet device, including, for example, the helmet devices 200, 230, 250, 270, 400, 450, 470, 490, 500 and 550 described herein.

In at least some embodiments, the user can interact with the user interface 120 to modify user-determined settings, such as desired treatment time, or the total power of the emitters 102 or emitter groups, which may be selected to more optimally treat the user's amount of hair loss.

In some embodiments, a user can modify the modes of operation using the user interface 120 to enter user-determined settings of operation for the modes of operation. For example, the user can modify which emitters should be pulsed in the pulsed mode of operation, and/or can modify the rate of pulsing. Further, in some embodiments, the user can modify the width and/or pattern of the bands of activated emitters in the sequential mode of operation. Further, in some embodiments, the user can modify the treatment time associated with each mode of operation.

In some embodiments, the user can modify the modes of operation by connecting the brush device 100 to a computing device (not shown) and modifying the modes of operation by using the computing device's user interface. In embodiments where the user can modify the modes of operation using a computing device, the laser therapy module 121 comprises a communication module (not shown), such as a USB port, a parallel port, a serial port, a wireless radio and the like, for communication with the computing device, using a corresponding communication scheme as is known by those of ordinary skill in the art.

In alternative embodiments, the device settings and modes of operation can only be modified after the user enters a password, such that modification of the device settings is only carried out in consultation with a light-therapy physician or practitioner who knows the password. In such an embodiment, the user may enter a password before modifying the device settings. The password may be preset before the device is sold to customers, such that a purchaser of the device does not necessarily know the password for the device ahead of time but may be given the password in a device manual that may be sold with the device or an invoice or a separate communication once the device is purchased by the user. In such embodiments, the variable control module 126 includes a processor, an ASIC, an FPGA, discrete electronics or the like, collectively referred to as a controller, which can be programmed or otherwise configured to modify the settings and modes of operation according to manipulation of the user interface 120 by a user. The variable control module 126 may be provided with software instructions to implement the functionality.

The power module 128 is coupled to the power switch 122 and the power indicator 124. The power switch 122 may be a switch, a button or any other means known to those of ordinary skill in the art such that the user can manipulate the power switch 122 to activate or deactivate the brush device 100. In some embodiments, the user interface 120 may be coupled to the power switch 122, such that the user activates the brush device 100 by using the user interface 120. The power indicator 124 generally indicates if the brush device 100 is activated and the power indicator may be an LED or other suitable element as is known to those skilled in the art.

In some embodiments, the power module 128 comprises a self-contained power unit, such as a battery, which may or may not be rechargeable. In such embodiments, the power indicator 124 may also be implemented to indicate the remaining power stored in the self-contained power unit with a visual, audible or tactile cue.

Alternatively, in some embodiments, the power module 128 may be coupled to an external power source 130, such as an electrical outlet as in laser therapy module 131, shown in FIG. 5.

Alternatively, in some embodiments, the power module 128 may comprise a self-contained power unit, and is additionally connectable to an external power source 130, e.g. for charging a self-contained power unit.

It should be understood that in these various embodiments, the power module 128 further comprises electrical components to adjust the voltage that is supplied to power the device. For example, a transformer and/or switched mode power supply may be used. Furthermore, it should be understood that the power module 128 further comprises electrical components to ensure that the brush device 100 is safe for use. For example, fuses, current limiters, insulative material and the like may be used as is known to those skilled in the art.

Reference is now made to FIGS. 6A-6C, which illustrate steps in a mode of operation of the brush device 100 for which there is sequential activation of emitters 102 during use. As explained above, the user interface 120 allows a user to select a mode of operation of the brush device 100. FIGS. 6A, 6B and 6C illustrate successive steps of a mode of operation of the brush device 100 in which a band of light due to the activated groups of laser emitters 143, 143′, and 143″ propagates through, the rows of emitters 102. In this mode of operation, the variable control module 126 controls the emitters 102 such that adjacent emitters 102 in one or more rows or emitter groups of one or more rows of emitters 102 are sequentially activated in successive steps so that a band of emitted light propagates across the active surface 106 of the brush device 100. For example, FIGS. 6A-6C show bands of emitters 143, 143′, 143″, respectively, in which three rows of emitters are being activated during a given step. In FIGS. 6A-6C, the activated emitters are shown with reference numerals 140 and are not shaded while the inactive emitters are shown with reference numerals 142 and are coloured black.

In alternate embodiments, there may be different patterns of emitted light due to a particular sequential activation of the emitters 102 or emitter groups. For example, in some embodiments, two rows of emitters may be activated during a given step. Furthermore, in another alternative, more than one row of activated emitters may be activated during a step, where one row of activated emitters is not adjacent to another row of activated emitters. For example, two separated rows of emitters may be active with one inactive row of emitters in between. Further, in other embodiments, the number of rows of activated and deactivated emitters for one sequencing may be changed.

In alternative embodiments, there may be overlap in the active rows of emitters between adjacent steps. For example, while FIG. 6A shows that three rows of emitters are active in the first step and three adjacent rows are activate in the next step (FIG. 6B), in some alternative embodiments only one of the three rows may be deactivated and only one adjacent row may be activated between adjacent steps. For example, in an alternative embodiment, the first step of the sequence may be as is shown in FIG. 6A, but in the second step, the bottom row of emitters for emitter group 143 is inactive, the top two rows of emitters for emitter group 143 are active and the bottom row of emitters for emitter group 143′ is active. Further steps may proceed in the same manner such that at least one active light band propagates across the active surface 106 during use.

In some alternative embodiments, the band of activated emitters may instead comprise a column of emitters or a diagonal line of emitters.

In some alternative embodiments, the number of rows in a band of active emitters or the time between steps can be modified by the user.

It should be noted that for the mode of operation described with respect to FIGS. 6A-6C and the various alternatives just described, this mode of operation generally provides less energy to the treatment surface than if the emitters are continuously activated, such that it is best suited for thin-haired individuals to prevent overstimulation, Additionally, because the groups of activated emitters move in sequential bands instead of random groupings, the user treatment surface may be given more time to recover between receiving doses of light energy.

Reference is now made to FIGS. 7-11, which illustrate an embodiment of a laser therapy helmet device 200 having a housing 200h. The housing 200h may be a headband as illustrated in FIG. 7. Alternatively, the housing 200h may be a cone or dome that covers the area of the head that produces hair for a normal hair bearing person. As in the laser therapy brush device 100, described above, the laser therapy helmet device 200 comprises an emitter array housing 216, an emitter array active surface 218, a plurality of laser emitters 102 and a plurality of bristles 214. FIG. 8 shows a plan view of emitter array active surface 218. The emitter array active surface 218 is a concave surface of the emitter array housing 216, as best shown in FIGS. 9-11.

The emitters 102 and the bristles 214 are mounted to the emitter array housing 216 such that the bristles 214 extend outwardly from the emitter array active surface 218 towards the user's, treatment surface (when the device 200 is in use), and such that the emitters 102, when activated, emit coherent light away from the active surface 218 towards the user's treatment surface (when the device is in use). The bristles 214 and the emitters 102 may be arranged in the same manner as the bristles 104 and the emitters 102, described in relation to FIGS. 1 to 3.

In most embodiments the bristles 214 have the same construction as the bristles 104. The bristles 214 may be suitably varied with regard to the bristles 104 to ensure optimum parting of the user's hair for the helmet device 200, such as by lengthening the bristles 214 to longer than about ½ an inch, for example. In addition, or in the alternative, the helmet device 200 may comprise more emitters 102 than the brush device 100 because the helmet device 200 may comprise a larger active surface 218. In the illustrated embodiments, the helmet device 200 comprises about thirty emitters 102. However, in other embodiments, a different number of emitters 102 may be used.

Although the emitter array housing 216 and the emitter array active surface 218 are shown as being rectangularly shaped, the emitter array housing 216 and the emitter active surface 218 may be shaped differently in alternative embodiments. For example, at least one of the emitter array housing 216 and the emitter active surface 218 may have a circular, oval, or hexagonal shape, or may extend along only half of the arc of the helmet 200, for example. Some example embodiments are described in relation to FIGS. 15A-B.

The laser therapy helmet device 200 also comprises a mount 205, a coupling 203 and an actuator 202. The coupling 203 provides for movement of the emitter array housing 216. In the example embodiment of FIG. 7, the coupling 203 is a rotational coupling. In other example embodiments, the coupling 203 may be a pivot coupling. The helmet mount 205 may be coupled to the emitter array housing 216 or may be integral with the emitter array housing 216, depending on the particular embodiment. In the illustrated embodiments, the emitter array housing 216 is rotationally coupled to the mount 205 with the rotational coupling 203. During use, the actuator 202 is operable to rotate the emitter array housing 216 relative to the mount 205 and the user's head 201 (along the path 206 shown in FIG. 7). While the illustrated embodiments show that the actuator 202 is coupled to the emitter array housing 216, in alternative embodiments the actuator 202 may be coupled to the mount 205.

There may be a multitude of embodiments of the rotational coupling 203 and the actuator 202. For example, in at least some embodiments, the rotational coupling 203 may be a shaft with appropriate rotational bearings mounted to the mount 205 and/or the emitter array housing 216. Furthermore, in at least some embodiments, the actuator 202 may be an electric motor with an output shaft coupled directly or indirectly to the rotational coupling 203.

In some embodiments, the helmet 200 also comprises spacer(s) 208 and/or a chin strap 210. The spacer(s) 208 are mounted to the mount 205 to evenly support the helmet 200 on the user's head 201. Although the spacer(s) 208 are shown positioned at the front and back of the mount 205 in FIG. 7, the spacer(s) 208 may also or alternatively be positioned at other locations on the mount 205. The chin strip 210 is coupled to the mount 205 and is looped around the user's chin in use to stably and releasably support the helmet 200 on the user's head 201. In alternative embodiments, other straps or restraints may be used to support the helmet device 200 on the user's head 201.

In some alternative embodiments, the spacer(s) 208 may be suitably varied to support the helmet 200 on other body parts of the user, for example the user's shoulders or back.

In some alternative embodiments, the spacer(s) 208 may be varied to support the helmet 200 on the users clothing or a mount worn by the user.

In some alternative embodiments, the spacer(s) 208 may be shaped differently to comfortably support the helmet 200 on the user's head 201.

Reference is now made to FIG. 12, which illustrates another example embodiment of a laser therapy helmet device 230 that uses a guide track to rotate the emitter array housing 216 relative to the helmet mount 205 during use. The helmet 230 comprises a guide track upper edge 234, a guide track lower edge 236, a guide wheel 240, and an actuator 232. In the illustrated embodiment, the actuator 232 is coupled to the emitter array housing 216, and the guide wheel 240 is rotationally coupled to the actuator 232. The mount 205 is coupled to the guide track upper edge 234 and the guide track lower edge 236. In use, the guide track upper edge 234 and the guide track lower edge 236 serve as a guide track for the guide wheel 240. In use, the actuator 232 rotates the guide wheel 240 moving it along the guide track such that the emitter array housing 216 rotates relative to the mount 205 and the user's head 201. The guide wheel 240 is positioned such that it has sufficient friction with the guide track so that the emitter array housing 216 rotates about the mount 205 when the guide wheel 240 is rotated by the actuator 232.

In some embodiments, the guide wheel 240 comprises guide wheel teeth (not shown) while the guide track upper edge 234 and the guide track lower edge 236 comprise grooves (not shown) that releasably mate with the guide wheel teeth in use. The guide wheel teeth cause relative rotation of the emitter array housing 216 about the mount 205 when the guide wheel 240 is rotated by the actuator 232.

In an alternative embodiment, the actuator 232 and the guide wheel 240 may be coupled to the mount 205 and an alternate guide track (not shown) may be affixed to the emitter array housing 216.

In alternative embodiments, the emitter array housing 216 comprises a guide flange (not shown) and the mount 205 comprises a vertical mount surface (not shown). The guide flange may extend from the emitter array housing 216, providing an approximately vertical mounting surface. The vertical mounting surface is an approximately vertical surface of the mount 205 whereupon the guide track may be mounted. In such embodiments, the guide wheel 240 and/or the actuator 232 are mounted to the guide flange. This embodiment provides for smooth rotation of the emitter array housing 216 relative to the mount 205, because the guide track and the guide flange are not angled, such that increased stability is achieved. In alternative embodiments, the guide track may be mounted to the guide flange, and the guide wheel 240 and/or actuator 232 may be mounted to the vertical mount surface.

Reference is now made to FIG. 13, which illustrates an example of another embodiment of a laser therapy helmet device 250 that has a guide wire 254 which is used to rotate the emitter array housing 216 relative to the mount 205. In the illustrated embodiment, the helmet device 250 comprises a guide wire 254, an actuator 252, a guide wheel 256 and tie downs 260, 262. In the illustrated embodiment, the actuator 252 is coupled to the housing 216. The guide wheel 256 is rotationally coupled to the actuator 252 and is also rotationally coupled to the guide wire 254. The guide wire 254 is coupled to the mount 205 by the tie downs 260, 262 which prevent the guide wire 254 from moving relative to the mount 205 by more than a nominal distance. In use, when the actuator 252 rotates the guide wheel 256, the guide wheel 256 rotates relative to the guide wire 254, causing the emitter array housing 216 to rotate relative to the mount 205 and the user's head 201.

In some embodiments, the emitter array housing 216 comprises a biasing means (not shown) for biasing the guide wire 254 against the guide wheel 256, such that there is sufficient friction between the guide wheel 256 and the guide wire 254 so that when the guide wheel is rotated, the emitter array housing 216 rotates relative to the mount 205. In some embodiments, the biasing means comprises a spring-biased member extending from the emitter array housing 216 and providing a biasing force to rotatably couple the guide wheel 256 to the guide wire 254.

In some alternative embodiments, other elements may be used to prevent motion of the guide wire 254 with respect to the mount 205. For example, in some alternative embodiments, the guide wire 254 may be mounted within a groove (not shown) in the mount 205, wherein the groove is shaped to receive the guide wheel 256 and to encourage smooth rotation of the emitter array housing 216 about the mount 205.

In some alternative embodiments, the guide wire 254 may be circumferentially looped around the guide wheel 256.

In some alternative embodiments, the guide wire 254 may be coupled to the housing 216, while the actuator 252 and the guide wheel 256 may be coupled to the mount 205. In such embodiments, the guide wire 254 may be rigid and may not be circumferentially looped around the guide wheel 256.

In some alternative embodiments, the guide wheel 256 may be rotationally coupled to the guide wire 254 with a plurality of teeth and grooves.

In some alternative embodiments, the guide wheel 256 may comprise guide wheel teeth (not shown) and the guide wire 254 may comprise a plurality of guide wire grooves (not shown) for receiving the guide wheel teeth.

Reference is now made to FIG. 14 which illustrates an example of another embodiment of a laser therapy helmet device 270 that is ground-coupled. The helmet device 270 comprises a mount coupling structure 271, an actuator 272 and an emitter array housing 216. The mount 275 may directly or indirectly rest on a ground or another load-bearing surface, such that the weight of the helmet device 275 is substantially supported by the mount 275 instead of by the user's head 201. However, it is not necessary that the mount 275 be rigidly coupled to the ground. For example, mount 275 may be a sliding mount or a rolling mount.

In some alternative embodiments, the actuator 272 may be rigidly coupled to the mount coupling structure 271 and rotationally coupled to the emitter array housing 216, such that in use, the actuator 272 rotates the emitter array housing 216 about the mount 275 and the user's head 201.

In some alternative embodiments, the actuator 272 may be rigidly coupled to the emitter array housing 216 and rotationally coupled to the mount coupling structure 271.

In some alternative embodiments, the actuator 272 may be rigidly coupled to the mount 275 and rotationally coupled to the mount coupling structure 271, such that in use, the actuator 272 rotates the mount coupling structure 271 about the mount 275,

In some alternative embodiments, the actuator 272 may be mounted to the mount 275 and force from the actuator 272 may be translated through the mount coupling structure 271 to the emitter array housing 216 through a translation element such as, but not limited to, a belt system, for example.

Reference is now made to FIGS. 15A and 15B, which illustrate examples of alternative embodiments of emitter array housings for use with laser therapy helmet devices. In the illustrated embodiments, the emitters 102 are mounted to the emitter array housings 286 and 296 such that when activated the emitters 102 emit coherent light away from the active surfaces 288 and 298, respectively, towards the user's treatment surface (when in use). In FIG. 15A, the emitter array housing 286 is shaped as a cross. In FIG. 15B, the emitter array housing 296 includes an emitter array active surface 298 having a plurality of parallel emitter active surfaces. Although the emitter array active surface 298 illustrates three active surfaces, there may be embodiments with more active surfaces.

Referring now to FIG. 16A, shown therein is a perspective view of an example alternative embodiment of a laser therapy helmet 400 with an automated pivoting emitter array housing 416. The emitter array housing 416 is implemented such that it has a front-back orientation and pivots in a side-to-side manner. The laser therapy helmet 400 comprises actuators 402 and 404 that are coupled to the emitter array housing 416 at first and second ends via pivot couplings 403 and 405 respectively. The pivot couplings 403 and 405 are located at the midpoint of the lower portion of the rear of the housing 200h and the midpoint of the lower portion of the front of the housing 200h respectively. The pivot couplings 403 and 405 are aligned along a pivot axis Al that lies in the sagittal plane so that the emitter array housing 416 automatically moves in a side-to-side motion. The helmet 400 further comprises limit members 408 on an inner surface of the housing 200h that limit the motion of the emitter array housing 416 so that it does not hit the user's ear. The limit members 408 may be the same as the spacers 208.

The actuators 402 and 404 are coupled to the emitter array housing 416 such that during use the actuators 402 and 404 cause the emitter array housing 416 to move along an arc by pivoting about pivot points A1 away from the sagittal plane where one arc 400pr is towards the user's right ear and the other arc 400p1 is towards the user's left ear. Accordingly, the inner surface of the emitter array housing 416 that faces the user is concave and shaped to match the contour of the user's head so that the emitter array housing 416 remains at a similar distance away from the user's head as it pivots about the user's head. The actuators 402 and 404 may be motors with output shafts that act as the pivot couplings for the emitter array housing 416 and the couplings 403 and 405 have appropriate pivoting bearings mounted to the housing 200h and/or the emitter array housing 416 or are directly coupled to the output shafts of the motors.

The actuators 402 and 404 can be configured to rotate by a certain amount so that the emitter array housing 416 pivots about a predefined arc, such as +/−90 degrees with respect to the sagittal plane. In some embodiments, the actuators 402 and 404 can be dynamically controlled to rotate at a certain speed and by a certain amount as described in further detail below which in turn cases the emitter array housing 416 to pivot at a desired speed and travel a desired distance.

Referring now to FIG. 166, shown therein is a perspective view of another example alternative embodiment of a laser therapy helmet 450 with an automated pivoting emitter array housing 466. The emitter array housing 466 is implemented such that it has a side-to-side orientation and pivots in a front-back manner. Like the laser therapy helmet 400, the laser therapy helmet 450 comprises actuators 452 and 454 that are coupled to the emitter array housing 466 at first and second ends via pivot couplings 453 and 455 respectively. However, the pivot couplings 453 and 455 are located at the midpoints of the sides of the housing 200h near the user's ears. The pivot couplings 453 and 455 are now aligned along a pivot axis A2 that lies in the user's frontal plane so that the emitter array housing 466 automatically moves in a front-to-back or back-to-front motion. The helmet 450 further comprises limit members 458 that limit the motion of the emitter array housing 466 so that it does not hit the user's forehead or nose when the emitter array housing 466 pivots forward along arc 450pf or it does not hit the back of the user's neck (i.e. the nape area) when the emitter array housing 466 pivots backwards along arc 450pb. The limit members 458 are disposed on an inner surface of the housing 200h. The actuators 452 and 454 may be implemented and operated similarly as the actuators 402 and 404 except that the emitter array housing 466 can now pivot forward to about −90 degrees or backward about +135 degrees with respect to the user's frontal or coronal plane.

It should be noted that in alternative embodiments for either helmet 400 or helmet 450 that only a single actuator and with a single coupling can be used at one end of the emitter array housing while a pivot member, such as a rotational bearing or a pin and socket, for example, can be used at the other end to facilitate the pivoting motion. This leads to a less expensive and potentially more robust implementation since not as many moving components are required.

In alternative embodiments, the actuation mechanism for the pivoting embodiments may be operated differently. For example, there may be a guide track or a guide wire that is oriented along the intended arc of motion for the emitter array housing in which case the emitter array housing comprises a coupler that is coupled to an actuator that moves along the guide track or the guide wire.

For example, referring now to FIG. 16C shown therein is a perspective view of an example of another alternative embodiment of a laser therapy helmet 470 with an automated pivoting emitter array housing 486. The motion of the emitter array housing 486 is similar to that of emitter array housing 416 except a different arrangement of components are used to implement the pivoting motion. The laser therapy helmet 470 comprises pivot members 471 at the lower mid-portion of the front and back of the housing 200h about which the emitter array housing 486 pivots. The pivot members 471 may be rotational bearings or a pin and socket coupling. The helmet 470 also comprises limit members 478 to limit the amount of pivoting done by the emitter array housing 486 as described previously for helmet 400. The helmet 470 further comprises a guide wire 474 which is used to pivot the emitter array housing 486 relative to the housing 200h. The helmet 470 also comprises an actuator 472, a guide wheel 473 and tie downs 475. The actuator 472 can be a motor. The guide wheel 473 is rotationally coupled to the actuator 472 and is also rotationally coupled to the guide wire 474. The guide wire 474 is coupled to the housing 200h by the tie downs 475 which prevent the guide wire 474 from moving relative to the housing 200h by more than a nominal distance. In use, when the actuator 472 rotates the guide wheel 473, the guide wheel 473 rotates relative to the guide wire 474, causing the emitter array housing 486 to pivot sideways to the left 470p1 or pivot sideways to the right 470pr about the user's head. In some embodiments a biasing means (not shown) may be used to bias the guide wire 474 against the guide wheel 473, such that there is sufficient friction between the guide wheel 473 and the guide wire 474 so that when the guide wheel 473 is rotated, the emitter array housing 486 pivots in a smooth fashion relative to the housing 200h. There may be alternative embodiments with respect to the implementation of the guide wire 474 and the guide wheel 473 as was described for the helmet 250.

In an alternative embodiment, referring now to FIG. 16D, shown therein is a perspective view of an example of another alternative embodiment of a laser therapy helmet 490 with an automated pivoting emitter array housing 496. The motion of the emitter array housing 496 is similar to that of emitter array housing 416 except a different arrangement of components are used to implement the pivoting motion. The laser therapy helmet 490 comprises pivot members 491 at the lower mid-portion of the front and back of the housing 200h about which the emitter array housing 496 pivots. The pivot members 491 may be implemented similarly to pivot members 471. The helmet 490 also comprises limit members 498 to limit the amount of pivoting done by the emitter array housing 496 as described previously for helmet 470. The helmet 490 further comprises a guide track 494 which is used to move the emitter array housing 496 relative to the housing 200h in a pivoting motion along arc 490p1 to the left and arc 490pr to the right. The helmet 490 also comprises an actuator 492 and a guide wheel 493. The actuator 492 can be a motor. The guide wheel 493 is rotationally coupled to the actuator 492 and is also rotationally coupled to the guide track 494. The guide track 494 is securely mounted to the housing 200h. The guide track comprises side edges that are wide enough to be engaged by the guide wheel 493 so that when the actuator 492 rotates the guide wheel 493, the guide wheel 493 rotates within the guide track 494 and has sufficient friction with the guide track 494 causing the emitter array housing 496 to pivot sideways to the left 490p1 or pivot sideways to the right 490pr about the user's head. There may be alternative embodiments with respect to the implementation of the guide track 494 and the guide wheel 493 as was described for the helmet 230. For example, in an alternative embodiment, the guide wheel 493 comprises guide wheel teeth (not shown) while at least one side edge of the guide track 294 comprise grooves (not shown) that releasably mate with the guide wheel teeth in use which causes the emitter array housing 296 to rotate when the guide wheel 493 rotates.

It should be noted that in alternative embodiments of helmets 470 and 490, the guide wire and guide track, respectively, and some of the other components may be oriented differently so that the emitter array housings 486 and 496 follow the pivoting motion of the emitter array housing 466 of helmet 450.

In alternative embodiments, there may be two or more emitter array housing that move relative to the user's head. For example, referring, now to FIG. 17, shown therein is a perspective view of an example embodiments of a laser therapy helmet 500 with two automated pivoting emitter array housings 516aand 516b. The emitter array housing 516a is configured to pivot in a front-back motion along a rear portion of the user's head. The emitter array housing 516b is configured to pivot about the front, top and side portion of the user's head.

The actuators 502a and 504a are coupled to opposite ends of the emitter array housing 516a via couplings 503a and 505a, respectively, at lower left and right rear sides of the helmet 500 in a similar fashion as helmet 450 so that the emitter array housing 516a pivots backwards along arc 500pf and forwards along arc 500pb. Limit members 508a limit the backward and forward motion of the emitter array housing 516a for safety purposes in case either of the actuators 502a and 504a malfunction. In alternative embodiments, the motion of the emitter array housing 516 may be facilitated using a guide wire or a guide track similar to helmets 470 and 490. In some alternative embodiments only one actuator with a coupling is used and a pivot member is used on the other side as described for helmets 400 and 450.

The actuator 502b is coupled at a front end of the emitter array housing 516b via coupling 503b in a similar fashion as the front end of emitter housing 416 for helmet 400 so that the emitter array housing 516b pivots along an arc 500ps in a side to side fashion to cover the front, top and sides of the user's head. Limit members 508b limit the motion of the emitter array housing 516b for safety purposes in case either of the actuators 502 and 504 malfunction. In alternative embodiments, the other end of the emitter array housing 516b may be coupled to a guide wire or a guide track similar to helmets 470 and 490 to provide for more secure motion of the emitter array housing 516b.

It should be noted that in an alternative embodiment the positions of the light emitter housings 516a and 516b can be reversed such that the light emitter housing 516b is located at the rear of the helmet and is configured to pivot from left to right and back again about the user's sagittal plane while the light emitter housing 516a can be disposed at the top front of the helmet and is configured to pivot front and back from the front of the user's head to their crown.

Referring now to FIG. 18, shown therein is a perspective view of an example alternative embodiment of a laser therapy helmet 550 with three automated pivoting emitter array housings 566a, 566b and 566c. The emitter array housing 566a along with actuators 552a and 654a, couplings 553aand 555a and limit members 558a operate in a similar fashion as the emitter array housing 516, actuators 502 and 504, couplings 503 and 505 and limit members 508 of helmet 500. However, the emitter array housing 566b is configured to move along an arc to cover the left side, left top half and left front half of the user's head while being limited by limit members 558b and the emitter array housing 566c is configured to move along an arc to cover the right side, right top half and right front half of the user's head while being limited by limit members 558c. One end of the emitter array housing 566b is coupled to actuator 552b via coupling 553b and the actuator 552b provides rotational force to cause the emitter array housing 566b to move along arc 550p1. In a similar manner, one end of the emitter array housing 566c is coupled to actuator 552c via coupling 553c and the actuator 552c provides rotational force to cause the emitter array housing 566c to move along arc 550pr.

Reference is now made to FIGS. 19-20, which illustrate a laser therapy module 300 that may be used with the various helmet devices shown herein. The laser therapy module 300 comprises emitters 102; a variable control module 126 coupled to the emitters 102 for controlling the emitters 102, and a power module 128 coupled to the variable control module 126 for powering the emitters 102. The laser therapy module 300 is generally mounted in the emitter array housing 216. The laser therapy module 300 further comprises at least one of a user interface 120, a power switch 122, and a power indicator 124. The laser therapy module 300 is thus similar to the laser therapy module 121, which is described in relation to FIG. 4, in that the elements that are in common for both the laser therapy module 121 and the laser therapy module 300 operate in a similar manner. The laser therapy module 300 further comprises an actuator control module 302 for controlling the actuator 202. The actuator 202 is generally powered by the power module 128. Each of these components may be mounted on one or more circuit boards as was similarly described for the brush device 100.

The actuator control module 302 can selectively activate, deactivate, or otherwise control actuator 202. In some embodiments, the actuator control module 302 is operable to vary the output of actuator 202, such that in use, the actuator control module 302 can vary the rotational speed and acceleration between the emitter array housing 216 and the mount 205. The actuator control module 302 may comprise a processor, an ASIC, a hardware controller and the like, which can be programmed or otherwise configured to provide control signals. Accordingly, the actuator control module 302 may be provided with software instructions to implement certain functionality.

Further, in some embodiments, the actuator control module 302 may be operable to control the output of the actuator 202 depending on the mode of operation of the device, as selected at the user interface 120.

Additionally, in some embodiments, the user can interface with the user interface 120 to control the actuator 202. For example, in some embodiments, the user may be able to use the user interface 120 to modify the force exerted by the actuator 202 (and thus the rotational speed of the housing 216) in the modes of operation by providing an appropriate input to the actuator control module 302.

In the embodiments in which the emitter array housing is automatically moveable, the user interface 120 is coupled to the actuator control module 302 and may be used by the user to select at least one of a speed of motion for the emitter array housing and a degree of motion for the emitter array housing. This advantageously provides more accuracy in terms of the amount of time that light treatment delivered to the user's head as the emitter array housing moves about the user's head. For example, in embodiments in which the emitter array housing may be rotated relative to the helmet, the actuator control module 302 can control how quickly the emitter array housing rotates about the user's head. In addition, or alternatively, in these “rotating embodiments”, the actuator control module 302 can control the degree of rotation allowing the user to select rotation along a small arc from 0 degrees to 180 degrees with respect to the nasal midline of the user's head (i.e. the nasal midline is a vertical plane that is centred on the user's nose and is also called the median or sagittal plane). Alternatively, in “pivoting embodiments” in which the emitter array housing may be pivoted along an arc about the user's head, the actuator control module 302 can control how quickly the emitter array housing pivots about the user's head. In addition, or alternatively, to speed control in these “pivoting embodiments”, the actuator control module 302 can also control the range of motion of the emitter array housing along the arc to be anywhere between 0 degrees to +/−90 degrees about the nasal midline of the user's head or between 0 degrees to +90 degrees towards the front of the user's head and between 0 degrees and about 135 degrees towards the back of the user's head (in this case the plane defining the 0 degree mark is a vertical plane that runs through the centre of the user's ears which is also referred to as the frontal or coronal plane).

Although, the laser therapy module 300 was described in relation to the helmet device 200, the elements of the laser therapy module 300 may be varied in position or structure to operate with the actuators 232, 252 or 272, or the mount 276 of the helmet devices 230, 250 and 270. For example, FIG. 20 illustrates an example embodiment in which the laser therapy module 300 is mounted within the mount 205 instead of within the emitter array housing 216.

Reference is now made to FIGS. 21A-21C, which illustrate an example for several steps of a sequential activation mode of operation of any of the laser therapy helmet devices described herein, in which at least one band of light propagates across the rows of laser emitters by activating certain rows of emitters in successive steps. The following description is with respect to laser therapy helmet device 200, for ease of illustration. As with the brush device 100 described in relation to FIGS. 6A-6C, sequential activation mode of operation of the helmet device 200 is best suited for thin-haired individuals to prevent overstimulation. In this example sequential mode of operation, the helmet device 200 comprises a group of activated laser emitters 310 and a group of deactivated laser emitters 312. The implementation of the sequential mode of operation illustrated in FIGS. 21A-21C is somewhat similar to the implementation of the sequential mode of operation described in relation to FIGS. 6A-6C. In the sequential mode of operation illustrated in FIGS. 21A-21C two bands of light may be generated by separating two activated emitters groups 316 and 318 by a group of inactive emitters. While, the sequential mode of operation shown in FIGS. 21A-21C is described in relation to the helmet device 200, the pattern in this mode of operation may also be used with one of the brush devices described herein. However, the higher number of emitters 102 on the helmet device 200 may facilitate different patterns of sequential activation that are not possible with a brush device. For example, the thickness of the light bands may be smaller for a brush device compared to a helmet device. Also, for the helmet device, the sequential mode of operation may use a less than three or more than three rows of active emitters for the light bands. The helmet devices 230, 250 or 270 may also comprise a sequential mode of operation.

Reference is now made to FIGS. 22A-22B, which illustrate an example of a mode of operation of the laser therapy brush device 100 and the laser therapy helmet device 200 in which a combination of continuous emitters and pulsing emitters are active.

This mode of operation may be applied to any of the other helmet devices described herein although the following description is with respect to helmet device 200 for ease of illustration. This therapy mode is best suited for individuals with moderate hair loss to create effective bio-stimulation while simultaneously decreasing the possibility of overstimulation. In the laser therapy brush device 100 and the laser therapy helmet device 200, the variable control module 126 controls the emitters 102 such that a plurality of the emitters 102 operate as continuous emitters 320 and at least one emitter 102 operates as a pulsing emitter 322. A continuous emitter is an emitter that is continuously powered, such that it continuously emits coherent light. A pulsing emitter is an emitter that rapidly switches (or is switched) from outputting coherent light to not outputting coherent light, which reduces the average output power from the laser emitter to the user's treatment surface. In the illustrated embodiments, pulsing may be achieved by rapidly switching laser emitters on and off.

There may be other embodiments that provide pulsing laser light emission from laser light emitters. For example, in some alternative embodiments, continuous laser emitters and an optical modulator may be used, such that the optical modulator is operated to only let light pass through for successive short periods of time during operation. In other alternative embodiments, at least one of Q switching, mode locking, cavity dumping, gain switching, and/or opto-electronic oscillators, for example, may be used to provide pulsing laser light emission.

It some embodiments, the helmet devices 200, 230, 250, 270, 400, 450, 470, 490, 500, or 550 may also comprise a mode of operation in which one or more combinations of continuous and pulsing emitters are active.

Reference is now made to FIG. 23, which shows a block diagram of an example embodiment of a laser emitter module which may be used with any of the various embodiments of the laser therapy brush device or the laser therapy helmet device described herein to selectively activate emitters 102 in order to carry out one of the modes of operation. The majority of these components have been described previously.

The control module 126 comprises a control unit 402 and a switching' network 404. Each of the laser emitters 102 may be directly coupled to the switching network 404 which is in turn coupled to the control unit 402. The control unit 402 may be implemented as a processor, an ASIC, a hardware controller and the like, which can be programmed or otherwise configured to provide control signals. Accordingly, the control unit 402 may be provided with software instructions to implement certain functionality depending on its implementation.

In this example embodiment, each of the emitters 102 may be independently wired to the switching network 404 to allow the control unit 402 to send control signals individually to the emitters 102 to have them operate according to a selected mode of operation. The switching network may be implemented using NMOS or CMOS transistors that operate as switches or using other components that are suitable to operate as switches such as diodes, for example.

The control unit 402 controls the operation of the various brush devices and helmet devices described herein. The control unit 402 provides control signals to the switching network 404 to activate or deactivate the emitters 102 according to a desired sequence or mode of operation. The user interface 120 allows a user to select the mode of operation, or in some embodiments to customize the operation of the brush devices or the helmet devices described herein.

For the various helmet devices described herein, the block diagram of FIG. 23 may be modified by adding a connection between the control unit 402 and the various actuators described herein (depending on the embodiment). Furthermore, in each of these cases, the actuator control module 302 may be implemented by the control unit 402.

Reference is, now made to FIG. 24, which shows a block diagram of the laser emitter module when the laser emitters are configured as groups of pulsed emitters 322 and continuous emitters 324. In this case, the control signals are provided from the control unit 402 and the switching network 404 so that it appears as if the emitters in the sets of pulsed emitters 322 are connected in parallel and the emitters in the set of continuous emitters 324 are connected in parallel. The control unit 402 may send control signals to the switching network 404 to activate or deactivate the sets of emitters 322 and 324, in conjunction with a timer. In other embodiments, other wiring or switches may be used to generate different activation patterns.

Reference is now made to FIG. 25, which shows a block diagram of the laser emitter module of FIG. 23 when the laser emitters 102 are grouped in rows for sequential activation. In this case, the control signals may be provided from the control unit 402 and the switching network 404 so that it appears as if the emitters in each row are connected in in parallel with one another but connected independently of the emitters in the other rows/columns. The control unit 402 and the switching network 404 operate to activate or deactivate the>rows or columns of emitters 102, optionally in conjunction with a timer,

In some of the pivoting and rotational embodiments, the mode of operation of the emitter array housing may be varied by the control unit 402 to deliver a different amount of light energy to the user's treatment surface as the emitter array housing automatically moves around the user's head. In such embodiments, at least one of the helmet embodiments described herein may incorporate a positional encoder to determine the location of the emitter array housing as it moves relative to the user's head and the control unit 402 controls the light treatment based on a user treatment protocol which specifies the amount of treatment which can include specifying one of more of light intensity, light pulse duration, light activation pattern, light wavelength, length of the treatment session and frequency of the treatment sessions per day or per week. This is advantageous since a particular amount of light treatment, also referred to previously as a mode of operation, can be selected based on an amount of hair loss experienced by the user in order to more effectively treat the hair loss for that particular user. This is beneficial since the user will have different amounts of hair at different locations on their head and, as described previously, hair blocks at least some light energy from reaching the user's treatment surface, and therefore if a user has less hair, it may be beneficial to select a mode of operation wherein less energy is output to the user's treatment surface in order to prevent overstimulation so the mode of operation can be automatically changed depending on the location of the head that the emitter array housing is facing. For example, one or more of the four modes of operation described previously may be used by the control unit 402 to control the light output of the emitter array housing.

The laser emitter module of any of the various embodiments described in accordance with the teachings herein may be configured such that all of the modes of operation are user-selectable on a single device. Accordingly, the laser emitter module may be configured such that the switch network is operable to send control signals to groups of emitters for sequential activation as in FIG. 24, and separately to pulsing and continuous emitters as in FIG. 25. Additionally, or alternatively, the laser emitter module may be operable to send control signals to individual emitters.

The various embodiments of the apparatuses described in accordance with the teachings herein may be used to implement a method of treating hair loss. Using a laser brush device or a laser helmet device, as taught herein, to treat hair loss may comprise at least the steps of: assessing the user's current hair loss; selecting one or more modes of operation based on the user's current hair loss to deliver an optimum amount of light energy to the treatment surface such that the use is tailored to the user's amount of hair loss; activating the device according to one of the one or more modes of operation and bringing it into contact or close proximity with the treatment surface such that the treatment surface receives coherent light from laser emitters provided on the device; and, either moving an active surface of the device across the treatment surface or keep the active surface stationary if the activation pattern is stationary or non-stationary, respectively, while the device is activated.

In some embodiments, it may be beneficial to use more than one mode of operation with the same user where different modes of operation may be used in different treatment sessions, for example,

In some alternate uses, during a single treatment session, a user may select a first mode of operation and later may select a second mode of operation part-way through the treatment session, such that the treatment session comprises a combination of modes of operation to provide an optimum amount of laser energy to the user treatment surface.

In some alternative embodiments, the emitter array housing does not comprise any bristles.

In some alternative embodiments, at least one emitter array housing has a flat surface.

Various embodiments of apparatus and devices have been described herein by way of example only. Furthermore, the apparatus and methods described herein may be used for the treatment of conditions other than alopecia, such as to treat scalp wounds, diabetic ulcers, and/or other ulcers to improve healing time. Various modifications and variations may be made to these example embodiments without departing from the spirit and scope of the embodiments, which is limited only by the appended claims which should be given the broadest interpretation consistent with the description as a whole.

### REFERENCES

Budd, D., Himmelberger, D, Rhodes, T., Cash, T. E., Girman, C. J., The effects of hair loss in European men: a survey in four countries, European Journal of Dermatology, Volume 10, Number 2, 122-7, March 2000, Cas cliniques, http//wwww.jle.com/e docs/00/01/89/92/article.phtml
Leavitt, Matt L. Follicle facts. In: Haber R S, Stough D B (eds) Hair transplantation. Elsevier Saunders, Philadelphia, 2006. p 189
Unger, Martin G., Low-Level Laser Therapy Is Now A Do-It-Yourself Treatment, press release of the International Society Of Hair Restoration Surgery, New York, Oct. 16, 2003. http://www.ishrs.org/press-release/low-level-laser-therapy-now-do-it-yourself-treatment.

We claim:

1. A laser therapy device comprising:
a helmet housing;
an emitter array housing moveably mounted to the helmet housing, the emitter array housing having an active surface being adapted to face a treatment surface of a user for treating hair loss during use;
a light therapy module positioned at least partially within the emitter array housing, the light therapy module having:
a plurality of emitters for emitting coherent light away from the active surface at a wavelength suitable for treating hair loss;
a variable control module coupled to the emitters for controlling the emitters; and
a power module coupled to the variable control module for powering the emitters;
at least one actuator coupled to the emitter array housing to move the emitter array housing; and
an actuator control module coupled to the at least one actuator and configured to control the at least one actuator to move the emitter array housing with respect to the helmet housing,

2. The device of claim 1, wherein the actuator control module is configured to rotate the emitter array housing with respect to the helmet housing.

3. The device of claim 1, wherein the actuator control module is configured to pivot the emitter array housing along an arc.

4. The device of claim 3, wherein the actuator control module is configured to pivot the emitter array housing about a sagittal plane of a user of the laser therapy device.

5. The device of claim 3, wherein the actuator control module is configured to pivot the emitter array housing about a frontal plane of a user of the laser therapy device.

6. The device of claim 3, wherein the device comprises first and second, emitter array housings and first and second actuators coupled to the first and second emitter array housings respectively, wherein the first actuator is configured to moving the first emitter array housing in a front-back pivoting manner and the second actuator is configured to move the second emitter array housing in a side-to-side pivoting manner.

7. The device of claim 6, wherein the first emitter array housing is located at a rear portion of the helmet housing and the second emitter array housing is located at a front central portion of the helmet housing.

8. The device of claim 6, wherein the second emitter array housing is located at a rear portion of the helmet housing and the first emitter array housing is located at a front central portion of the helmet housing.

9. The device of claim 3, wherein the device comprises first, second and third emitter array housings and first, second and third actuators coupled to the first, second and third emitter array housings respectively, wherein the first actuator is configured to moving the first emitter array housing in a front-back pivoting manner at a rear portion of the helmet, the second actuator is configured to move the second emitter array housing in an arcing motion along the right side of the helmet housing and the third actuator is configured to move the third emitter array housing in an arcing motion along the left side of the helmet housing.

10. The device of claim 3, wherein the actuator comprises a motor and the device comprises a coupling for coupling an output shaft of the motor to the light emitter array housing.

11. The device of claim 3, wherein the actuator comprises a motor and the device comprises a guide wire that is disposed along a portion of the helmet housing defining a pivot arc along and a guide wheel that is coupled to the motor and to the light emitter array housing, and the guide wheel engages the guide wire wherein during use the motor rotates the guide wheel which moves along the guide wire causing the light emitter array to pivot along the arc.

12. The device of claim 3, wherein the actuator comprises a motor and the device comprises a guide track that is disposed along a portion of the helmet housing defining a pivot arc along and a guide wheel that is coupled to the motor and to the light emitter array housing, and the guide wheel engages the guide track wherein during use the motor rotates the guide wheel which moves along the guide track causing the light emitter array to pivot along the arc.

13. The device of claim 1, wherein the actuator control module is configured to vary at least one of the speed and direction of movement of the emitter array housing.

14. The device of claim 1, wherein the device comprises a control unit that is configured to control the emitter array housing to operate according to one of a plurality of modes of operation for delivering varying amounts of light energy to the treatment surface of the user, wherein a mode of operation is chosen based on an amount of hair loss experienced by the user to more effectively tailor the treatment to the user's amount of hair loss.

15. The device of claim 14, wherein the control unit is configured to deliver varying amounts of light energy according to the position of the emitter array housing relative to a portion of the treatment surface of the user.

16. The device of claim 15, wherein the control unit is configured to deliver a larger amount of light energy when the position of the emitter array housing faces a portion of the treatment surface of the user with more hair than other portions of the treatment surface of the user.

17. The device of claim 15, wherein the control unit is configured to deliver a smaller amount of light energy when the position of the emitter array housing faces a portion of the treatment surface of the user with less hair than other portions of the treatment surface of the user.

18. The device of claim 14, wherein in a mode of operation the variable control module is configured to sequence the emitters, wherein sequencing the emitters comprises sequentially activating and deactivating rows of emitters, such that at least one band of light energy is emitted along the rows of emitters.

19. The device of claim 18, wherein the sequencing comprises deactivating at east one row of emitters and activating at least one adjacent row of emitters.
